(19) United States
(12) Patent Application Publication
(10) Pub. No.: US 2008/0167571 A1
(43) Pub. Date: Jul. 10, 2008

(54) DETERMINATION OF TREATMENT RESULTS PRIOR TO TREATMENT OR AFTER FEW TREATMENT EVENTS

(76) Inventor: Alan Gevins, San Francisco, CA (US)

Correspondence Address: Alan Gevins, SAM Technology, Inc., 425 Bush Street, Fifth Floor, San Francisco, CA, 94108, US

(21) Appl. No.: 11/957,156

(22) Filed: Dec. 14, 2007

Related U.S. Application Data
(60) Provisional application No. 60/870,829, filed on Dec. 19, 2006.

(51) Int. Cl. A61B 5/0476

(52) U.S. Cl. 600/544

(57) ABSTRACT

Disclosed are methods and data on determining response to a drug or other therapy prior to administration by obtaining a direct neurocognitive brain function measurement; obtaining an indirect neurocognitive brain function measurement; and, assessing the direct neurocognitive brain function measurement and the indirect neurocognitive brain function measurement collectively to obtain a response determination preferably the predictive value of the collective assessment is greater than a predictive value obtained from the separate predictive values for the direct and indirect measurements. Also disclosed are methods of determining dosage of a drug comprising administering a drug; comprising obtaining a direct neurocognitive brain function measurement; obtaining an indirect neurocognitive brain function measurement; and, assessing the direct neurocognitive brain function measurement and the indirect neurocognitive brain function measurement collectively to obtain a dosage, preferably the predictive value of the collective assessment is greater than a predictive value obtained from the separate predictive values for the direct and indirect measurements; optionally, additional cycles of obtaining and assessing indirect and direct measurements are performed.

### GOVERNMENT SUPPORT

This invention was made with government support under grant R44 NS042992, awarded by the National Institute of Neurological Disorders and Stroke. The Government has certain rights in the invention.

### FIELD OF THE INVENTION

The present invention relates to medical decision-making, including diagnosis, prognosis, prophylaxis, and treatment using direct and indirect measures of brain function All documents referred to herein are fully incorporated herein for all purposes.

### BACKGROUND OF THE INVENTION

Various medical conditions, normal and pathologic, present neurological and cognitive manifestations. Previously, attempts have been made to make decisions about such conditions using direct brain measures. For example, the diagnosis of epilepsy may involve an EEG (electroencephographic) exam a direct brain measure. This is one of the earliest and widely applied uses of EEG and QEEG (Quantitative EEG). Various books, chapters, articles and patents have been directed toward the detection of seizures and brain wave patterns associated with the diagnosis of epilepsy. For example: Savit et al USP Application 20060200038 relates to detection of ictal onset and seizures in epilepsy. Two EEG recordings are taken at different brain locations. Similarly U.S. Pat. No. 6,061,593 to Fischell et al attempts, at least 5 seconds before seizure, to detect the onset of clinical symptoms using a d-c shift in EEG voltage.

In Monastra et al U.S. Pat. No. 6,097,980 a QEEG is used to diagnose patients for Attention Deficit Hyperactivity Disorder (ADHD). A single cranial electrode records brain wave activity which is analyzed into various frequency bands while the subject has a fixed gaze. These measures are compared to comparable measures while the subject reads, listens or draws. The comparative data from one subject is then compared to similar data from a normal group.

Moreover, EEG has been used to measure the side effects and effectiveness of certain drugs. In Suffin et al USP Applications 20050251419 and 20030144875, a psychiatric patient's EEG is compared to a database of similar patients to predict the neurological effect of a drug. USP Application 20040152995 to Cox et al uses EEG inconsistencies to diagnose and test treatment of persons with attentional or cognitive impairments. Cox et al collect digitized EEG data during initial and later periods, i.e. during the same day, while the subject performs “cognitive tasks”. The QEEG power change distance between the two periods is compared to a control group database. The “cognitive tasks” are watching a video or reading, neither of which requires that subjects make a response to verify that they were paying attention or to gauge the quality of their attention In Greenwald et al USP Application 2003081821 a bispectral or higher order spectral QEEG measure is used to predict medication effectiveness and also to measure response to medication from a pre-medication baseline. In Devlin et al USP Applications 20050216071 and 20050043774 QEEG signals from a patient undergoing treatment, i.e. neurostimulation, are analyzed for various features and indices. Pretreatment indices are used to predict response to treatment. Changes in the indices are used to judge efficacy of treatments.

The inherent weakness of any method or system that attempts to predict or characterize the effect of a treatment using only direct measures of brain function is that all such measures reflect many factors including some that may be irrelevant to the treatment effect being predicted or characterized. In principle, if one assembled a sufficiently large data base of patients to characterize the actual brain function measures that truly predict or characterize the treatment of interest, such irrelevant factors should cancel out. In practice, this is rarely if ever done because there are usually so many such irrelevant factors for any given treatment or condition that an impractically large sample of patients would be required.

Consequently, analyzed in isolation direct brain function measures can produce erroneous or misleading conclusions about an individual's clinical state and what treatment would be best for that individual unless other information about the patient's cerebral state is simultaneously considered. For instance, a mildly drowsy patient's EEG brain function measures reflect a low degree of overall brain activation that has the same general neuroelectric signal characteristics as mildly pathological brain function (i.e. increased widespread low frequency EEG power). Such signals of low alertness may have no relevance whatsoever to whether that patient has a brain dysfunction that is likely to respond to a particular medication or other treatment, for instance depression or amnestic mild cognitive impairment suggestive of early Alzheimer's disease.

To address the problem of confounding variables when direct brain measures are assessed in isolation, certain indirect measure have been included in analyses. When other information about the patient, called herein indirect brain function measures, are combined with the direct brain function measures, such erroneous conclusions can be avoided. An additional benefit of combining direct and indirect brain function measures is that the ability to recognize the medical condition or effect of treatment is increased. For instance, by combining such information about alertness with the direct measures of brain function, the effect of varying alertness may be factored into the predictive equation, and a patient's low alertness would not affect the determination of whether the patient has a brain function pattern associated with the likely future success or failure of a particular treatment. The inventor's prior patents (U.S. Pat. Nos. 5,295,491, 6,434,419, 6,947,790) addressed the issue of the insufficiency of direct brain function measures by themselves for characterizing how a patient reacted to a treatment by teaching how to combine direct brain function measures with one type of indirect brain function measure, namely measures of the subject's performance of attention demanding tasks. For instance, by combining direct measures of a subject's brain function with measures of the accuracy of a subject's task performance, higher sensitivity and specificity of detection of the neurocognitive effects of a variety of drugs was demonstrated, and it was possible to distinguish between cognitive impairment due to a drug's sedating properties from impairment due to a drug's neurotoxic effect by also including indirect brain function measures of alertness, e.g., in the form of electrophysiological measures of eye movements. To continue with an example of a drowsy patient, since low alertness is the primary symptom of patients with sleep disorders, the direct brain function measures associated with low alertness would be the relevant biomarkers rather than confounding variables in this instance. In order to determine whether a patient with another medical condition with brain function measures similar to those of low alertness, for instance a metabolic disorder that produced widespread signs of mildly pathological brain function also had a condition affecting alertness, an indirect measure of brain function would be required, in this case a metabolic biomarker.

While helpful, combining such task performance indirect brain function information with direct brain function measures was not possible in some health care settings. Also, the choice of which indirect measures of brain function to use is specific to the medical condition being tested; statistically good information for one condition may be irrelevant for another.

Thus, heretofore unmet needs have existed in the field for achieving statistically relevant assessment of patient conditions. Although the combination of certain direct and certain indirect measure has helped, some of the indict measurements may be inconvenient to obtain in some health care settings. Direct measures are often taken in radiology departments, and many indirect measures are obtained by health care professionals from other disciplines; in this situation, when direct and indirect measures are not obtained simultaneously, it requires coordination amongst the individual disciplines. If more streamlined indirect measure could be added to direct brain measures and still obtain quality results, this would be an improvement in the art. It also would be an improvement to the art to provide mathematical and algorithmic methods to quantitatively combine direct and a variety of indirect brain function measures.

The current invention overcomes issues in the prior art described above by combining direct with a variety of new types of indirect measures of brain function and facilitates neurocognitive analysis.

Moreover in the areas of medical decision-making, such as prognosis and diagnosis, there are longstanding needs to obtain better predictive information. As set forth below, the invention is used to predict whether, or to characterize how, a patient will respond to a treatment. Thus, we have combined indirect and direct brain function measures to accurately predict a drug's effect before the drug is taken, and have used the invention to closely gauge doses that were eventually clinically determined by a physician.

### BRIEF DESCRIPTION OF THE DRAWINGS

FIG. 1. A schematic diagram of the system used in the present invention.

FIG. 2. Cognitive Neurophysiological Effects of Taking 200 mg of Carbamazepine Daily for 30 Days. Dark bars are the four subjects out of 28 for whom the unintended cognitive and neurophysiological effects (poorer cognitive performance and EEG signs of neurotoxicity) of taking 200 mg of carbamazepine daily for 30 days were more than 1 S.D. worse than the average of the group of 28 subjects shown in the left-most bar.

FIG. 3. Predicting, Before Subjects Took the Drug, the Unintended Impairment of Cognitive Performance Due to Taking 200 mg of Carbamazepine Daily for 30 Days: Six worst subjects compared to group. Non-drug baseline direct and indirect brain function measures predict the approximate severity of decrement for five of six subjects out of 28 with a 15% or greater decline in neuropsychological test performance (Symbol-Digits Modality Test) after taking 200 mg of carbamazepine daily for 30 days. The predicted effect is shown in the right bar of each pair, while the actual effect due to taking the drug is shown in the left bar of each pair. The predicted and actual effects on the group of 28 subjects is shown on the left-most pair of bars.

FIG. 4. Predicting, Before Subjects Took the Drug, the Unintended Impairment of Cognitive Performance Due to Taking 200 mg of Carbamazepine Daily for 30 Days. Whereas the overall effect of this drug on the cognitive performance of the group of 28 subjects was negative (left-most bar), some subjects exhibited only mild effects, if any, while others' cognition was quite debilitated. Non-drug baseline direct and indirect brain function measures significantly (p<0.001) predicted the substantial range of individual differences in severity of decrement in neuropsychological test performance (Symbol-Digits Modality Test) after taking 200 mg of carbamazepine daily for 30 days. The predicted effect is shown in the right bar of each pair, while the actual effect due to taking the drug is shown in the left bar of each pair. For 10 of the 13 subjects whose response to taking the drug for 30 days was worse than the group mean, the predicted response was also worse than the mean response, while for 11 of the 15 subjects whose response was better than the group the predicted response was also better than the group.

FIG. 5. Predicting the Optimal Dose of a Drug After Test Doses. The dose of methylphenidate selected by the algorithm combining direct and indirect brain function measures (“selected dose”) was within 5 mg of the dose selected by a pediatric psychiatrist specialist in 12 of 13 pediatric patients being treated for attention deficit hyperactivity disorder.

FIG. 6. Cognitive Neurophysiological Effects on 29 Subjects of Taking 300 mg of Topiramate Daily for 30 Days. The average response across the group is shown in the left-most (striped) bar. The 10 individual subjects on the left side of the graph (solid bars) for whom the unintended cognitive and neurophysiological effects (poorer cognitive performance and EEG signs of neurotoxicity were considered to be “bad responders,” while the 10 individual on the right side of the graph (dotted bars) were considered “OK responders” for the prediction analysis.

FIG. 7. Predicting, Before Subjects Took the Drug, the Unintended Impairment of Cognitive Performance Due to Taking 300 mg of Topiramate Daily for 30 Days. Non-drug baseline direct and indirect brain function measures predict the approximate severity of decrement for the 10 “bad responders” from FIG. 6 after taking topiramate daily for 30 days. The predicted effect is shown in the right bar of each pair, while the actual effect due to taking the drug is shown in the left bar of each pair. The predicted and actual effects on the group of 29 subjects is shown on the left-most pair of bars. All 10 “bad responders” were predicted to have a below average response to topiramate.

### SUMMARY OF THE INVENTION

An efficient, objective method and system using direct and indirect measures of neurocognitive function or brain function is described. The present invention has been used for quantifying treatment effects prior to administration of the treatment itself, i.e., from a “pre-treatment baseline.” In addition, the invention has been used after initial treatment is undertaken to predict outcome and to more successfully manage the ongoing treatment regimen. The present invention is useful in a wide variety of medical settings, including but not limited to an office setting, a clinic setting, or a hospital setting.

The invention comprises using combinations of neurological, genetic and behavioral biomarkers to determine a reaction to a treatment before the treatment is administered, and/or to evaluate the effect of the treatment after it is administered, such as to refine a dose. The invention is employed as part of the successful treatment of diseases or conditions that directly or indirectly affect human neurocognitive performance, or with those conditions whose treatments affect neurocognitive performance. The invention is also used to determine whether drugs have a significant positive effect on delaying or improving the symptoms of a disease or condition, especially during clinical trials for drug approval and subsequent marketing. The invention is also used to predict and evaluate long lasting changes in overall neurocognitive function following training and educational programs.

Indirect brain function information is helpful for reducing confounding variables, and can increase sensitivity and specificity of direct brain function measures. For example, such indirect brain function information can be an independent measure of the patient's alertness, for instance from video or electrophysiological measures of eye closures and slow drifting eye movements and/or from self-reported or observed alertness scales. For instance, information about the presence of a genetic marker that predisposes a patient to Alzheimer's disease, such as the aPoe4 gene, or information about brain structure, such as ventricle size or hippocampal volume, or information about the presence of amyloid plaques and neurofibrillary tangles, can be combined with the direct measure of brain function signals characteristic of early amnestic mild cognitive impairment to increase the sensitivity and specificity of early detection of the presence of the disease.

In one embodiment the invention comprises a method of predicting response to a drug prior to drug administration comprising: obtaining a direct neurocognitive brain function measurement; obtaining an indirect neurocognitive brain function measurement; and, assessing the direct neurocognitive brain function measurement and the indirect neurocognitive brain function measurement collectively to obtain a drug response prediction, whereby the predictive value of the collective assessment is greater than a predictive value obtained by adding separate predictive values for the direct and indirect measurements.

In another embodiment the invention comprises a method of determining dosage of a drug with a linear dose-response curve, comprising steps of: administering a drug; obtaining a direct neurocognitive brain function measurement; obtaining an indirect neurocognitive brain function measurement; and, assessing the direct neurocognitive brain function measurement and the indirect neurocognitive brain function measurement collectively to obtain a dosage, whereby the predictive value of the collective assessment is greater than a predictive value obtained by adding separate predictive values for the direct and indirect measurements.

In another embodiment, the invention comprises a method of determining dosage of a drug with a nonlinear dose-response curve to achieve a specified response, comprising steps of: administering a first test dose of the drug; calculating a dose-response value for the test dose; subsequently, administering a second test dose of the drug; calculating a dose-response value for the second test dose; calculating the slope between the responses to the two doses; extrapolating from the slope to a reach a specified response level; and identifying the dose that corresponds to that response level.

The direct neurocognitive brain function measures can comprise EEG, MEG, fMRI, PET and fNIR measures. In the case of EEG measures, direct measures can comprise both measures from the background EEG such as, but not limited to, power spectral measures and measures from stimulus-registered or response-registered evoked potentials such as, but not limited to, CNV, N100, N200, P200, P300, N400, Slow Wave and Response-related Potentials amplitude and peak latency.

The indirect neurocognitive brain function measures can comprise measures of psychometric or attention-demanding tasks, physiological measures, physical/anatomical measures, genetic measures, and chemical measures, or any measure that provides indicia of neurocognitive function. Psychometric or attention-demanding tasks include, but are not limited to, accuracy and reaction time, self reported or externally observed measures of the subject's affective, cognitive and alertness condition Physiological measures include those of physiologic or autonomic arousal such as, but not limited to, heart rate, respiration and skin conductance. Measures of CNS or brain structure include but are not limited to, volumetric measures of brain areas and ventricles, assessments of lamination or myelination assessment of plaques or mass formation. Genetic measures include but are not limited to, the presence of genes or gene products associated with particular pathologies or physiologies. Chemical measures include those of body fluids that characterize metabolism, metabolism of a substance or some physiologic health or disease state.

In one embodiment, the direct brain function measurement and the indirect brain function measurement elicit data on the same physiological functions as set forth in an example represented in Table 3; in another embodiment the physiological functions are selected from the group consisting of: attention regulation, memory, alertness regulation, regulation of other neurocognitive functions, regulation of sensory or motor functions, and regulation of mass neuronal synchronization.

Table 3 sets forth findings or results from Examples herein; Table 3 uses the alphanumeric numbering used for results in each Example. Table 3 is grouped by functional category and sets forth findings at various levels of generality; these levels are each an embodiment of the invention. This Table exemplifies and does not limit the invention. For a given Example, other embodiments, e.g., at other levels of generality or combining elements at various levels of generality will be apparent to those of skill in the art. In particular it is appreciated by those skilled in the art that the direct and indirect assessment set forth in Table 3 reflect particular physiologic traits; these physiologic traits can be measured via other direct or indirect modalities in accordance with the invention as well.

As appreciated by those skilled in the art, the variables in Table 3 and their exact or relative weightings are not unique representations of the neurophysiologic attentional processes or their behavioral manifestations assessed in a particular example. Other variables, with other relative and absolute weightings, that characterize the subject's performance and the neural regulation of such performance in brain regions can also be extracted using the same methodology on different sets of data. Examples of such alternative variables are described in the art such as in the inventor's prior patents and scientific publications referred to herein. Thus, the choice, combination and weighting of the variables do not merely reflect the variance in the particular data that were analyzed. The respective variables for an example represented in Table 3 also characterizes other treatments, drugs or classes of drugs that affect a patient, e.g., by the same route, mechanism of action, chemical property or elicited effect.

Other objectives and features of the present invention will be apparent from the following detailed description, taken in conjunction with the accompanying drawings and claims. All documents referred to herein, including patents, applications, articles, documents, etc., are fully incorporated herein for all purposes.

### DEFINITIONS

“ABLs” indicates anticonvulsant blood levels

“ADHD is Attention Deficit Hyperactivity Disorder

“AED” indicates antiepileptic drug.

“ANOVA” indicates analysis of variance, a collection of statistical models and their associated procedures which compare means by splitting the overall observed variance into different parts.

“CBZ” refers to carbamazepine, also known by the tradename Tegretol™. CBZ is a tricyclic compound, and an iminostilbene. CBZ is effective in treating, e.g., depression bipolar depression, neuralgia and seizures. CBZ is closely related to phenytoin. The ureide moiety present in most antiseizure drugs is also present in CBZ. The mechanism of action of CBZ is similar to phenytoin; it blocks sodium channels at therapeutic concentrations and inhibits high frequency firing of neurons in culture. CBZ also acts presynaptically to decrease transmission. CBZ interacts with adenosine receptors. It inhibits the release and reuptake of norepinephrine from brain synaposomes. CBZ may potentiate the post-synaptic effects of GABA. It is not sedating in its usual therapeutic range. Its absorption is erratic or non-linear after oral administration. More information on its chemistry, mechanism of action, clinical uses, effects, pharmacokinetics, therapeutic levels, dosing, drug interactions, toxicity and related drugs are available in the art, e.g., Basic & Clinical Pharmacology, 9^{th} ed., Katzung editor, (Lange McGraw, 2004); and, Goodman & Gilman's The Pharmacological Basis of Therapeutics, 11^{th} ed., Brunton editor, (McGraw, 2006), each of which are specifically incorporated by reference herein for these purposes.

“Cerebral capability” is the totality of a subject's brain state measured with a combination of direct and indirect brain function measures.

“Direct brain function measure” is any direct measurement of cerebral neuroelectric, neuromagnetic or neurometabolic activity, for instance with EEG, MEG, fNIR, fMRI, MRI spectroscopy or PET. Direct measures of brain function are made according to various art-accepted testing protocols. Direct brain function testing comprises obtaining data when patients: are passively awake or asleep, passively receive repetitive simplified sensory stimulation (e.g. trains of light flashes, tones, or electrical pulses), passively receive naturalistic sensory stimulation (e.g. watching TV, listening to music, receiving a massage), or while they actively perform attention-demanding tests that are either scored or not scored, each according to methodologies known in the art. The terms, “direct brain function measure,” “direct neurocognitive brain function measure” and “direct measure” are synonyms unless the context clearly indicates otherwise.

“EEG is an abbreviation of electroencephalogram, a direct brain function measure of the mass electrical activity of the brain.

“fMRI” is an abbreviation of functional magnetic resonance imaging, a direct brain function measure of the metabolic activity of the brain.

“fNIR” is an abbreviation of functional near-infrared imaging, a direct brain function measure of the metabolic activity of the brain. fNIR is an emerging spectroscopic neuroimaging method for measuring the level of neuronal activity in the brain. The method is based on the neurovascular coupling theorem which says that there is a relationship between metabolic activity and oxygen level (oxygenated hemoglobin) in feeding blood vessels.

“Genetic measures” may include a disease-specific gene and/or a biochemical abnormality thought to be genetically determined or influenced. Current examples include genetic measures for which there is a widely available test (e.g., tests of phenylketonuria or sickle-cell disease, measuring of cholesterol or lipoprotein levels) while others relate to genes that predict severe and untreatable neurological disease (e.g., Huntington's disease) or that suggest vulnerability to such a disease (e.g., Alzheimer's disease).

“Indirect brain function measure” is any measure, other than a direct brain function measure, which provides information relevant to characterizing an individual's cerebral capability, including without limitation, information about brain structure, e.g., as obtained from MRI, CT scans or x-rays, information from genetic measures, information from bodily fluids, information about a patient's behavior from task performance data, psychometric data, self-report data, third party assessment or clinical scales. The terms, “indirect brain function measure,” “indirect neurocognitive brain function measure” and “indirect measure” are synonyms unless the context clearly indicates otherwise. Information from task performance data is included in this term, and can be obtained as described in the inventor's U.S. Pat. No. 5,295,491; 6,434,419; or 6,947,790. In the case of an indirect brain function measure that comprises measures of cognitive task performance, cognitive functions required by the tasks can comprise simple or complex forms of attention including transient or sustained attention, selective or divided attention, preparatory, executive or feedback updating attention. The cognitive functions can also include various forms of memory including immediate working memory, episodic memory and long-term memory, receptive and expressive language, and more complex executive functions such as reasoning. At one extreme, the simplest cognitive task used during simultaneous collection of physiological signals is to ask a subject to follow an uncomplicated instruction such as to keep still with eyes open or closed until told that the recording is completed. During such period, various auditory, visual or somatosensory stimuli can be delivered with no requirement that the subject overtly respond to such stimuli. Examples of simple and complex cognitive tasks are described in Gevins and Smith, 2000, in Gevins et al, 1998, 1997, 1996, 1995, in McEvoy, Smith and Gevins, 2000, 1998, in Smith, McEvoy, and Gevins, 1999, and in Ilan, Smith, Gevins, 2004. Embodiments of sustained attention, working and episodic memory tasks are described in the examples set forth below and in the aforementioned scientific publications.

“LEV” refers to levetiracetam, also known by the tradename Keppra™. LEV is a piracetam analog, it is a pyrrolidine. Its kinetics are linear. The synaptic vesicle protein SVZA has been shown to be a target of LEV (Lynch et al, 2004). Information on its chemistry, mechanism of action, clinical uses, pharmacokinetics, therapeutic levels, effects, dosing, drug interactions, toxicity and related drugs are available in the art, e.g., Basic & Clinical Pharmacology, 9^{th} ed., Katzung editor, (Lange/McGraw, 2004); and, Goodman & Gilman's The Pharmacological Basis of Therapeutics, 11^{th} ed., Brunton editor, (McGraw, 2006), each of which are specifically incorporated by reference herein for these purposes.

“MEG” refers to magnetoencephalogram, a direct brain function measure of the magnetic component of the brain's electromagnetic activity.

“MCI” indicates mild cognitive impairment (MCI). Amnestic MCI is a precursor to Alzheimer's Disease.

“MPH” indicates methylphenidate, also known by the tradename Ritalin™ Methylphenidate is a sympathomimetic drug. MPH is a piperidine derivative and is an amphetamine variant. Its pharmacologic properties are essentially the same as amphetamines. It is very similar to pemoline (Cylert™) in its pharmacologic effects. Dexmethylphenidate (Focalin™) is the d-threo enantiomer of racemic MPH. It is used for ADHD and narcolepsy. Information on MPH's chemistry, mechanism of action, clinical uses, pharmacokinetics, therapeutic levels, effects, dosing, drug interactions, toxicity and related drugs are available in the art, e.g., Basic & Clinical Pharmacology, 9^{th} ed., Katzung editor, (Lange/McGraw, 2004); and, Goodman & Gilman's The Pharmacological Basis of Therapeutics, 11^{th} ed., Brunton editor, (McGraw, 2006), each of which are specifically incorporated by reference herein for these purposes.

“MRI is magnetic resonance imaging, an indirect brain function measure of brain structure.

“Neurocognitive” refers to brain function processes related to cognition, as well as to the subjective and objective manifestation of such processes.

“Normative population” is a sample (the minimum number to be included in the normative population depends on the heterogeneity of the population and on the number of age cohorts) that will allow for the assembly of a statistically relevant decision about a criterion. The results from the normative sample are used to compare the test results of a given test subject against the normative population and allow a statistically relevant assessment to be made.

“PET” indicates positron emission tomography, a direct brain function measure of the metabolic activity of the brain.

“Primary measures” are computed from the direct and indirect brain function data and comprise:
1) measures encoding information about brain structure from MRI, CT scans or x-rays; information from genetic measures; information from measures of patient chemistries, e.g., from blood, urine or cerebrospinal fluid; or, information about a patient's behavior from self-report data or clinical scales.
2) measures encoding information about task performance scores such as the mean, standard deviation and variability of the subject's accuracy and reaction time to each task trial, or simply the binary variable encoding whether or not the subject complied with the task instructions;
3) measures encoding information about the ongoing EEG such as the power and peak frequency of the subject's EEG or MEG delta, theta, alpha, beta and gamma band signals recorded over parietal, prefrontal temporal, central and occipital cerebral cortical brain regions;
4) measures encoding information about the EEG evoked potential component time registered to a stimulus or response such as the amplitude and peak time of the subject's Contingent Negative Variation, N100, N200, P200, P300, N400, P600, Slow Wave and Movement Potentials;
5) measures encoding information about slow and fast horizontal eye movements and eye blinks such as the magnitude of the subject's physiological signal power recorded near the eyes, and parameters characterizing eye movements and blinks output by eye-tracking and eyelid tracking equipment;
6) ratios of certain primary measures in paragraphs 2-5, for instance alpha plus beta divided by delta plus theta EEG power, or response accuracy divided by reaction time;
7) ratios or differences of each of primary measures enumerated above in paragraphs 3 and 4 between different locations on the scalp; or,
8) measures between different locations on the scalp of time series interdependency such as covariance, correlation, coherence or mutual information of the EEG or evoked potential time series enumerated above in paragraphs 3 and 4;
9) measures encoding information about fMRI and PET signal intensity in voxels or regions of interest, fMRI time series of signal intensity for voxels or regions of interest, principal components analysis, independent components analysis, covariance analysis, coherence analysis of the aforementioned time series.

“QEEG” is a quantitative electroencephographic exam.

“Related drug” indicates a drug that is chemically, structurally or mechanistically similar to a particular drug. Accordingly, in view of such similarities, the related drug and the particular drug perform similarly either in vivo or in vitro. Information on a drug of interest such as chemistry, mechanism of action, clinical uses, pharmacokinetics, therapeutic levels, effects, dosing, drug interactions, toxicity, and drugs related thereto are available in the art, e.g., Basic & Clinical Pharmacology, 9^{th} ed., Katzung editor, (Lange/McGraw, 2004); and, Goodman & Gilman's The Pharmacological Basis of Therapeutics, 11^{th} ed., Brunton editor, (McGraw, 2006), each of which are specifically incorporated by reference herein for these purposes.

“Secondary measures” comprise:
1) differences, ratios or other comparisons of the primary measures between pairs of task conditions, e.g.:
   a) between two simple tasks such as eyes-open and eyes-closed,
   b) between a simple task and a more attention-demanding task,
   c) between two more attention-demanding tasks, or
   d) between easy and more difficult versions of the same more attention-demanding task; and
2) differences or ratios of the primary measures or of secondary measure #1 between initial and subsequent repetitions of a task in the same session.

“SMDT” indicates Symbol Digits Modality Test

“Task performance data” comprises data to characterize or score the capability of the subject to perform tasks that require conscious awareness, for instance the mean, standard deviation and variability of the subject's accuracy and reaction time to each trial of an attention-demanding task, or whether and how well a subject complied with instructions given during the direct brain function test such as keep your eye s open or closed, watch the video, listen to the music, etc.

“SWAN” indicates the Strengths and Weaknesses of ADHD-symptoms and Normal-behaviors assessment.

“TOP” refers to topiramate, also known by the tradename Topomax™. Topiramate is an antiepileptic, antiseizure drug. TOP is a sulfamate-substituted monosaccharide. It blocks repetitive firing of cultured spinal cord neurons, as do phenytoin and CBZ. It appears to block voltage dependent sodium channels. It activates a hyper-polarizing K+ current. It appears to potentiate the effects of GABA, acting at a site different than benzodiazepines or barbiturates. It depresses the excitatory action of kainite on AMPA receptors. It is a carbonic anhydrase inhibitor. Its kinetics are linear. Information on topiramate's chemistry, mechanism of action, clinical uses, pharmacokinetics, therapeutic levels, effects, dosing, drug interactions, toxicity, and related drugs are available in the art, e.g., Basic & Clinical Pharmacology, 9^{th} ed., Katzung editor, (Lange/McGraw, 2004); and, Goodman & Gilman's The Pharmacological Basis of Therapeutics, 11^{th} ed., Brunton editor, (McGraw, 2006), each of which are specifically incorporated by reference herein for these purposes.

“WM” refers to “Working Memory,” and is the fundamental cognitive function of sustaining attention or maintaining conscious awareness on an internal representation of some external or internal object, event or abstraction.

### DETAILED DESCRIPTION OF THE INVENTION

One objective of the present invention is to provide neurological, genetic and behavioral biomarkers to predict patient outcome to a treatment. The invention is used to identify patients likely to have strong negative or positive neurocognitive effects, average neurocognitive effects, or mild or no neurocognitive effects to medical or other treatments including specific drugs within a drug class.

Thus, in one embodiment, the invention is used to determine whether a patient will have a strong positive therapeutic response, an undesirable negative response, an average response, or no response, to a drug or other treatment before taking the drug or receiving the treatment. The invention is also used to determine the optimal dose or treatment regime for treating a given patient after one or more test doses or test treatments.

In accordance with the present invention, one obtains one or more measures (both direct and indirect measures) of the quality of brain function from a normative population i.e., a population that contains the disease or trait of interest for medical remediation Respective data can also be obtained from a control population.

For example, to make pre-treatment assessments, direct and indirect brain function data is obtained from members of a normative population during a “pre-treatment” baseline; data is also obtained after members of the population receive the particular drug or treatment. The post-treatment brain function data is used to classify the normative population into responder classes for the particular drug or other treatment (nonresponder, strong responder, negative side effects, etc. as appreciated in the art). The pre-treatment brain function data, from each responder type is analyzed. The pre-treatment data is used to define patterns that correlate how each responder classification type responded to the treatment. These pre-treatment correlations of direct and indirect brain function now serve to predict how a patient will respond to a treatment.

In accordance with the invention a health care provider can readily compare a new patient's brain function assessments (direct and indirect) before and/or after receiving a particular drug or other treatment to the corresponding values from the normative population and classify the patient as, for example, a strong, average or mild/none responder. These findings are used to suggest a direction or change in treatment for the patient according to the schema in Table 1a-b.

TABLE 1a
Schema for choosing treatment using results from system.

| Pretreatment | |
|---|---|
| (predicted outcome) | Action to Take |
| Mild or no response | Try a different treatment |
| Average response | Consider this treatment |
| Strong response | If positive, try this treatment. |
| | If negative, try a different treatment |

TABLE 1b
Schema for adjusting treatment using results from system.

| After Treatment | Action to Take |
|---|---|
| Mild or no response | Consider increasing treatment parameters |
| | (dose) or try a different treatment |
| Average response | Continue treatment |
| Strong response | If positive, continue treatment. |
| | If negative, consider decreasing dose or try a |
| | different treatment |

To obtain patient data, the direct and indirect measures of brain function can be obtained separately or concurrently. In a preferred embodiment, such brain function measures are obtained from one round of testing. In alternative embodiments, two, three, or more test sessions take place with each session being compared to the normative and or control populations. In those embodiments comprising multiple test sessions, between-session change measures can be computed from the measures from the sessions; comparisons can be made to norms that reflect the normative amount of change between multiple test sessions.

Direct measures of brain function are made according to various art-accepted testing protocols. Direct brain function testing comprises obtaining data when patients: are passively awake or asleep, passively receive repetitive simplified sensory stimulation (e.g. trains of light flashes, tones, or electrical pulses), passively receive naturalistic sensory stimulation (e.g. watching TV, listening to music, receiving a massage), or while they actively perform attention demanding tests that are either scored or not scored, each according to methodologies known in the art.

The invention is used to assess treatments such as drugs, brain stimulation, psychotherapy and sensory, motor and cognitive rehabilitation therapies, surgery, radiation therapies and other treatments designed to diagnose or treat a condition that directly or indirectly affects cognition. Examples of such treatments comprise drugs including pharmaceutical preparations used to treat a wide range of conditions such as Attention Deficit Hyperactivity Disorder (ADHD), Alzheimer's Disease, Mild Cognitive Impairment (MCI), Depression, Schizophrenia, Bipolar Disorder, Anxiety, Migraine, Seizure, Epilepsy, Sleep Disorders, Parkinson's Disease, Multiple Sclerosis, Cancer, Diabetes, or any other disease/condition that has a direct or indirect impact on an individual's neurocognitive function. In addition, the invention is used to assess non-pharmaceutical treatments (e.g., the amount of current used in vagus nerve stimulation or deep brain stimulation), psychotherapy, electroconvulsive therapy (ECT) and other treatments in order to determine the best level of stimulation/treatment for a patient based on a single treatment session (e.g., for those with a linear administration-response curve/profile) or based on two, three or more treatment sessions (e.g., for those with either non-linear or linear administration-response curves/profiles).

The present invention is used in the diagnosis, prognosis, prophylaxis and treatment of a wide number of disease states and/or therapies. These disease/conditions and treatments include those in which an alteration of neurocognitive brain function is a byproduct of the disease/condition or of the treatment. Relevant diseases/conditions include but are not limited to: (1) Attention Deficit Hyperactivity Disorder (ADHD), (2) Alzheimer's Disease, (3) Mild Cognitive Impairment, (4) Depression, (5) Schizophrenia, (6) Bipolar Disorder, (7) Anxiety, (8) Migraine, (9) Seizure, (10) Epilepsy, (11) Sleep Disorders, (12) Parkinson's Disease, (13) Multiple Sclerosis, (14) Brain Injuries; (15) Cancer, (16) Diabetes, (17) any other disease/condition that has a direct or indirect impact on an individual's neurocognitive function Relevant therapies assessed with the invention include but are not limited to: anesthetics, oncologics (including chemotherapy, prescription medications and other cancer-fighting treatments), neurological and psychiatric medications, sleep medications, any other treatment that has a direct or indirect impact on an individual's cognition.

In addition to its use in the treatment of various conditions, the present invention is used to predict the effectiveness and adverse side effects of medications. In the case of drugs, the following classes of drugs are assessed in accordance with the invention: psychostimulants (e.g., for treating ADHD); anti degenerative brain disease drugs (including cholinesterase inhibitors), NMDA receptor antagonists, drugs that target amyloid-B peptides, drugs that boost nerve growth factor, anti-inflammatory drugs, etc. (for treating Alzheimer's Disease, amnestic Mild Cognitive Impairment, other dementias and other forms of mild cognitive impairment); antidepressants; antipsychotics; anxietolytics; anti-migraine drugs; anti-epilepsy drugs; anti-insomnia drugs and alertness increasing drugs (for treating sleep disorders); anti-pain drugs; anti-Parkinson's disease drugs; anti-multiple sclerosis drugs; and drugs of abuse.

Construction of a Normative Database

A key feature of the database construction is that both direct and indirect quantitative measures of each patient's brain function are included. Including direct and indirect measures allows these complementary types of information to be compared in order to predict or characterize how a patient will respond to a treatment.

The construction and application of normative data bases and equations to characterize post-treatment neurocognitive response and prediction of post-treatment response from a pre-treatment baseline can comprise a drug as the mode of treatment. The same process can be applied to the other treatments.

For example, for a drug or drug class of interest, one selects: one or more drugs representative of that class and one or more doses in the therapeutic range for each drug; a representative population of human subjects with appropriate diagnostic, age, gender, education, or genetic, characteristics; and a testing protocol including an appropriate choice of testing conditions. A relevant protocol is carried out in order to obtain baseline non-drug and post-drug direct and indirect brain function data, data appropriate for a condition and population of interest.

Assessment of the Normative Data

Experimental protocol designs include both crossover and parallel designs, with and without placebo controls. Quality control screening is performed to eliminate contaminated or otherwise invalid brain function data Numerical features and summary indices are computed from the direct and indirect data, as appropriate/relevant for the condition or treatment of interest.

Accordingly, upon weighing the direct and indirect data, one classifies members of the normative population into response classifications, e.g., positive, negative, strong, average, mild, none, side effect, etc. to the particular treatment. In one embodiment, equations are computed that combine the numerical features and summary indices for the direct and indirect data into a single summary score that distinguishes the post-treatment response to the drug from the pre-treatment baseline. However, the direct and indirect data can be collectively assessed without the need to produce a single score.

In view of the response classification, the pre-treatment data is analyzed in order to extract direct and indirect brain function patterns that correlated with how each responder class performed. This analysis generally produces predictive equations. In general one first computes numerical features and summary indices from the direct and indirect brain function data obtained from the experimental protocol and then trains and cross-validates a multivariate pattern classifier to distinguish between the strong and weak responder types, or to distinguish between each of the responder types. The numerical features and summary indices for the predictive equations preferably assess direct and indirect brain function measures that are sensitive to individual differences in brain function and genetic characteristics, and may optionally include the same brain function features as were used as inputs to compute the post-treatment equations, or other brain function measures.

When a normative population is tested in more than one round of testing, between-round change measures may be computed from the multiple sessions in a variety of permutations. Comparison can be made between respective rounds or from the baseline to an arbitrary round, etc. in order to obtain data that reflects the normative amount of change between multiple test sessions.

Construction of a Normative Database and Equations that Characterize Post-Treatment Response from Pre-Treatment Data

This section sets forth construction of a normative data base and equations to characterize post-drug neurocognitive response and prediction of post-drug response from non-drug (“pre-treatment”) baseline. For each drug class (or individual drug) of interest, the following are selected: one or more drugs representative of that class and one or more doses in the therapeutic range for each drug; a representative population of human subjects with appropriate diagnostic, age, gender and education characteristics; and a testing protocol including an appropriate choice of task conditions.

An experimental protocol is designed and executed to obtain baseline non-drug and post-drug data according to the testing protocol from the subject population Experimental protocol designs include preferably crossover or parallel designs, with or without placebo control conditions. A feature of the database construction is that both direct and indirect quantitative measures of each patient's brain function are included so that these complementary types of information can be combined in the analysis to predict or characterize how a patient will respond to a treatment.

The direct measures comprise those obtained in accordance with art-accepted modalities such as EEG, MEG, fNIR, fMRI, MRI spectroscopy or PET, etc. The indirect measures can comprise one or more of the following: information about brain structure from MRI, CT scans or x-rays, information from genetic measures, information from measures of blood chemistry, information about a patient's behavior from self-report data or clinical scales. Indirect measures which are information from task performance data may also be included as described in the inventor's U.S. Pat. Nos. 5,295,491, 6,434,419, 6,947,790. As described above, quality control screening is performed on the data obtained according to the experimental protocol to eliminate artifact-contaminated or otherwise invalid assessment data.

In an example with EEG assessment data: An appropriate set of primary and secondary summary measures are then computed based on prior knowledge of the effects of the chosen drugs or drug classes on EEG signals. If such prior knowledge is not available, a more general set of such summary measures are computed. For instance, it is well known that benzodiazepines increase beta band activity in the EEG, so a measure of beta band activity would be included when considering that class of drugs. Similarly, many anti-epilepsy drugs increase low frequency EEG power and such measures would be included in the analysis of such drugs. Lacking such prior knowledge, one would use more general EEG measures such as delta, theta, alpha and beta band power.

A well-determined equation (s) is obtained; a well-determined equation is one in which there were a sufficient number of subjects to extract class-distinguishing EEG variables that generalize to a statistically significant classification of a new sample of subjects. Since there are so many variables in any modality that directly measures brain function, it is often the case that equations are computed to distinguish classes of treatments with too few subjects given the number of variables, resulting in equations that are not well-determined. If the set of summary measures is too large to compute well-determined equations distinguishing non-drug and post-drug data given the number of subjects recorded, a smaller subset of the measures must be chosen. This can be accomplished by visual inspection and statistical tests of how the measures vary between non-drug and post-drug data, and/or preferably by the use of mathematical algorithms that systematically explore the measures to determine optimal or near optimal subsets of measures that distinguish non-drug and post-drug data.

The set of summary measures is the set from which an equation is computed that chooses and weights an optimal combination of a subset of the measures that best distinguishes between non-drug and post-drug conditions. The equation is computed using an appropriate statistical pattern recognition algorithm, preferably a neural network, a logistic or other type of regression, a multivariate divergence-based algorithm, or other type of multivariate dimensionality reduction and classification/prediction algorithm.

The output of such equation is, for example, a score that quantifies the normative post-treatment neurocognitive response to the drug. The statistical significance of the equation's ability to quantify the drug's effect is determined by reference to the appropriate binomial or multinomial distribution and is preferably represented in a receiver-operator characteristic curve. In a preferred embodiment the measure sub-set selection and pattern recognition analysis and statistical significance is validated through a jackknife procedure.

The subjects comprising the normative population are then sorted into post-drug responder classes, e.g., strong, average and mild/none responders to the particular drug or treatment based on the summary score, preferably using a statistically determined cutoff, for instance greater than one standard deviation above the mean population response for strong responders and greater than one standard deviation below for weak responders. Examples of the above procedure are described in Examples below.

Preferably, normative equations are computed with an analogous analytic strategy applied to distinguish the pre-treatment data of strong from weak responders, or to distinguish between each of the three or more response types. That is, using the database consisting of subjects who responded strongly or weakly to the drug or other treatment, the pre-treatment direct and indirect brain function measures of the strong and weak responders are analyzed in order to compute equations that can be applied to a new subject to predict, from that subject's pre-treatment brain function data, how strongly that subject will respond to the treatment.

The candidate set of summary measures used to train the predictive equations preferably includes features of the EEG from the above list of primary features that are known to vary between individual subjects, for instance those features described in Gevins and Smith, 2000 and U.S. Pat. No. 6,434,419, issued Aug. 13, 2002. The features optionally include genetic information and/or the same measures as were used as inputs to compute the post-treatment equations or were otherwise found to be affected by the drug or treatment.

Comparison of a Patient's Data with the Normative Database.

Upon assembly of the normative data, a new patient's brain function (direct and indirect) is assessed. These assessments are input into a predictive equation derived from the normative population. Accordingly, the invention provides an estimate of how the new patient is likely to respond to the drug, drug class or treatment of interest. The health care provider now chooses the best drug or dose of a drug for the patient based on the results, and prescribe that drug or dose according to the schema in Table 1a.

Optionally, the patient is tested again after he or she has initiated the prescribed drug, drug dose or treatment. This brain function data input into the post-treatment equation to assess how well the patient responded. If the patient has not responded satisfactorily, a comparable alternative or next best drug or dose or treatment is chosen from the predictive equation as set forth in Table 1b. Optionally the health care provider repeats the post-treatment assessment. In an embodiment of the invention in which alternative doses of the same drug can be prescribed, further adjustments in dose can be made by further application of the assessment steps building upon results with a prior test dose.

When a patient is tested more than once, each such test session can be compared with the normative population.

Alternatively, between-session change measures may be computed from the multiple sessions, and the comparison can be made to values derived from the normative population that reflect the normative amount of change between multiple test sessions.

Obtaining and Using Patient Data, Such as EEG Data

One embodiment of the invention comprises EEG brain function measures recorded during easy tasks and more attention-demanding psychometric tests. Analogous procedures in accordance with the invention are used when the other testing protocols enumerated above are employed; respective analogous procedures are used when using the other direct and indirect brain function measures enumerated above.

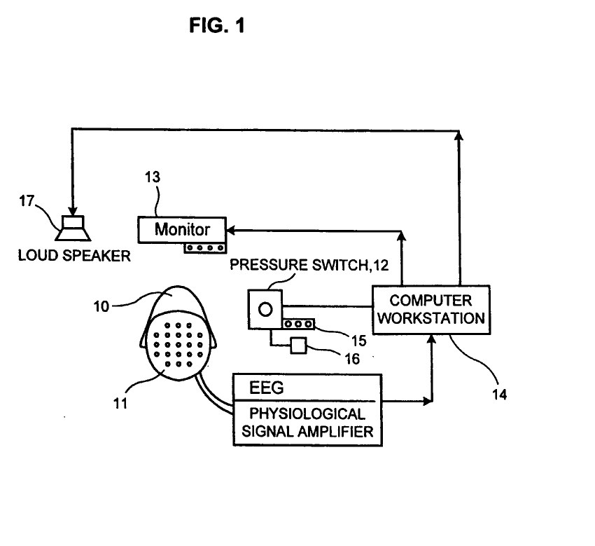

Referring to FIG. 1, a human subject 10, whose head is illustrated, wears a cloth hat 11, or headset having electrode leads which contact the scalp of the subject. The leads detect the subject's weak analog brain waves and also the electrical activity of his eyes and scalp muscles.

Suitable EEG hats are described in U.S. Pat. No. 5,038,782, issued Aug. 13, 1991, and in U.S. patent application Ser. No. 11/259,971 filed Dec. 12, 2005. The hat has preferably 1-32 independent electrodes, although more electrodes may be used. The brain waves are amplified, preferably as described in the U.S. Pat. No. 5,038,782 and artifacts detected and removed, for example, as described in U.S. Pat. No. 5,513,649 issued May 7, 1996.

In one embodiment: the subject's brain waves are recorded concurrent with an indirect brain function assessment; the indirect assessment comprises that the subject is presented with tasks that require one or more cognitive functions. In an alternative embodiment the indirect assessment tasks are not presented concurrently with the direct detection of the subject's brain waves or other physiologic signals; the direct signals may be recorded while the subject is drowsy or asleep.

In an embodiment where direct data is EEG data, referring to FIG. 1, the tasks are presented on the screen 13 of a computer monitor, and/or by a loudspeaker 17 connected to the digital computer workstation 14. The subject regards the monitor screen and/or listens to the loudspeaker and responds using a keyboard key 15, or alternatively a switch 12 or a joystick 16.

Following completion of the test session, the direct brain function measures and indirect brain function measures are analyzed to extract primary and secondary summary measures from the data in accordance with methodologies in the art, such as those described in Gevins, et al., 2002, 1998, 1997, 1996, in Gevins and Smith, 2000, 1999; in McEvoy, Smith, Fordyce, Gevins, 2006, in Smith, Gevins, McEvoy, Meador, Ray, Gilliam, 2006, in Ilan, Gevins, Coleman, ElSohly, de Wit 2005, in Ilan, Smith, Gevins, 2004, in Smith, McEvoy, Gevins, 2002, in Chung, McEvoy, Smith, Gevins, Meador, Laxer, 2002, and in Meador, Gevins, Loring, McEvoy, Ray, Smith, Motamedi, Evans, and Baum.

Primary Measures

Primary measures computed from the direct and indirect data generally include at least one of:
1) measures encoding information about brain structure from MRI, CT scans or x-rays; information from genetic measures; information from measurements of patient chemistries, e.g., from blood, urine or cerebrospinal fluid; or, information about a patient's behavior from self-report data (subject self-assessment of their condition including affective, cognitive and alertness assessments) or clinical scales.
2) measures encoding information about task performance scores such as the mean, standard deviation and variability of the subject's accuracy and reaction time to each task trial, or simply the binary variable encoding whether or not the subject complied with the task instructions;
3) measures encoding information about the ongoing EEG such as the power and peak frequency of the subject's EEG or MEG delta, theta, alpha, beta and gamma band signals recorded over parietal, prefrontal temporal, central and occipital cerebral cortical brain regions;
4) measures encoding information about the EEG evoked potential component time registered to a stimulus or response such as the amplitude and peak time of the subject's Pre-stimulus evoked potential (e.g., Contingent Negative Variation), Pre-P300 evoked potential (e.g., N100, N200, P200, N200), P300 evoked potential, Post-P300 evoked potential (e.g., N400, P600), Slow Wave and Movement Potentials;
5) measures encoding information about slow and fast horizontal eye movements and eye blinks such as the magnitude of the subject's physiological signal power recorded near the eyes, and the magnitude of eye-tracking and eyelid tracking equipment;
6) ratios of certain primary measures in paragraphs 2-5, for instance alpha plus beta divided by delta plus theta EEG power, or response accuracy divided by reaction time;
7) ratios or differences of each of primary measures enumerated above in paragraphs 3 and 4 between different locations on the scalp; or,
8) measures between different locations on the scalp of time series interdependency such as covariance, correlation, coherence or mutual information of the EEG or evoked potential time series enumerated above in paragraphs 3 and 4; and,
9) measures encoding information about fMRI and PET signal intensity in voxels or regions of interest, fMRI time series of signal intensity for voxels or regions of interest, principal components analysis, independent components analysis, covariance analysis, coherence analysis of the aforementioned time series.

Secondary Measures

Optionally, secondary measures are also computed. The secondary measure can include:
1) differences, ratios or other comparisons of the primary measures between pairs of task conditions, e.g.:
   a) between two simple tasks such as eyes-open and eyes-closed,
   b) between a simple task and a more attention-demanding task,
   c) between two more attention-demanding tasks, or
   d) between easy and more difficult versions of the same more attention-demanding task; and
2) differences or ratios of the primary measures or of secondary measure #(1) between initial and subsequent repetitions of a task in the same session.

From amongst the above primary and secondary measures, those measures required by a predictive equation previously derived from an appropriate normative population for a particular drug, class of drugs or therapy are entered into said equation in order to predict how the new patient will respond to the drug, drug class or therapy of interest.

If the response output of the equation for the new patient is more than a threshold amount, for instance one standard deviation, above the average response output for the appropriate normative population, this patient is deemed likely to have a strong reaction to the drug or class of drugs.

The response outputs are similarly computed for each normative equation of as many drugs or classes of drugs as the physician/health care provider deems relevant for the medical care of the patient. The physician can then choose the best therapy, drug or dose of a drug for the patient based on which equation had the most favorable indication of a desired response. The physician can then prescribe or modify that therapy, drug or dose according to the schema such as that set forth in Table 1a-b. An example of this process is described in Example 1.

The entire process of testing the patient and analyzing the data as described above can optionally be performed again after the patient has initiated the prescribed drug or dose, with the test data being input to an appropriate post-treatment equation to assess how well the patient responded. If the patient has not responded satisfactorily, the physician can choose and prescribe the next best drug or dose from the predictive equation and optionally repeat the test and post-treatment assessment. In the instance in which two or more doses of the same drug have been prescribed, further adjustments in dose can be made by extrapolation from the results with the prior test doses, as illustrated in Example 2.

### EXAMPLES

### Example 1

### Prediction of Drug Response

This example shows how the invention has been used to predict drug response prior to drug administration. Accordingly, from a non-drug baseline, the health care provider determines whether a subject will have a positive or an adverse neurocognitive response to the common anti-epileptic drugs. This study assessed the sensitivity of the present invention in evaluation of the neuropsychological and neurophysiological effects of the antiepileptic drug (AED) carbamazepine (CBZ).

One embodiment for use of the present invention is in the management of epilepsy (or other seizure disorders). Epilepsy is a common neurological condition that is characterized by recurrent unprovoked seizures. The seizures are transient signs and/or symptoms due to abnormal excessive or synchronous neuronal activity in the brain. It affects approximately 50 million people worldwide. Epilepsy is usually controlled, but not cured, with medication. At the present time the most common treatment of epilepsy is the use of medication, generally given orally.

The diagnosis of epilepsy often involves an EEG (electroencephographic) exam. This is one of the earliest and widely applied uses of EEG and QEEG (Quantitative EEG). Various books, chapters, articles and patents have been directed toward the detection of seizures and brain wave patterns associated with the diagnosis of epilepsy. Some of the US Patents literature is mentioned herein.

A patient, after being diagnosed as having epilepsy, may be treated with a wide variety of drugs and dosages, including Depakote™ (divalproex sodium), Neurontin™ (gabapentin), Lamictal™ (lamotrigine), Trileptal™ (oxcarbazepine), Keppra™ (levitiracetam) and others. The treating physician, before prescribing a specific epilepsy medication and its dosage, must consider a number of factors including the patient's age, general health condition, history of taking other drugs (especially other anti-epileptic drugs), the patient's racial classification, and the other medications being taken at the same time. The physician bases the prescription on his/her experience as well as scientific teaching on the subject.

Many of the myriad antiepileptic drugs (AEDs) currently on the market have similar efficacies in reducing seizures. Therefore, differential side effects play an important role in therapeutic decisions.

The purpose of the underlying study was to evaluate the neuropsychological and neurophysiological effects of the AED carbamazepine (CBZ), and the AED levitiracetam (LEV) in healthy subjects, employing a double-blind, two period crossover design (Meador et al., 2007). In accordance with the present invention, data at non-drug baseline sessions predicted which subjects would have the most negative effects of CBZ.

Subjects

A total of 28 healthy adult volunteers without history of neurological or psychiatric diseases completed the protocol (17 women; 11 men; mean age=33 years, range=18-51). All subjects remained free of centrally active prescription medications throughout the study. They also did not use over-the-counter medications or alcohol for 72 hours prior to each neurocognitive testing session.

Protocol

The study employed a double-blind, randomized, two period crossover design. Subjects were screened and tested at the non-drug baseline.

Subsequently, they were randomly assigned to receive either AED. Each AED was administered over eight weeks, which included a titration period and a one month maintenance period. Each AED treatment period was followed by a four day taper and a washout period for the remainder of the four weeks. Then, subjects were treated with the other AED for eight weeks followed by a final four week washout period.

CBZ was given at 200 mg/day for the first week, 200 mg/day bid for the 2nd week, then adjusted to midrange anticonvulsant blood levels (ABLs) at tid dosages. LEV was begun at 500 mg/day for two weeks, then increased to 500 mg bid for two weeks, then increased to 1000 mg bid.

Subjects underwent neuropsychological and neurophysiological testing on six occasions (i.e., two AED conditions and four non-drug conditions) over 25 weeks. Test sessions occurred at week one before drug administration, at week nine after four weeks of titration and maintenance on the first drug for four weeks, at week 21 after 4 weeks of wash out, 4 weeks of titration on drug 2 and 4 weeks maintenance on drug 2, and at week 25 after 4 weeks of washout.

Patient data was obtained in accordance with procedures set forth in U.S. Pat. Nos. 6,947,790; 6,434,419 and 5,295,491; in this embodiment EEG data was obtained simultaneously with the subject taking a test battery of attention demanding tasks, for example a set of psychometric tasks.

Data was obtained from a verbal episodic memory task consisting of a word presentation phase, during which subjects had to indicate whether each word contained 1 or 2 syllables, and a delayed recognition phase in which subjects had to indicate whether each word had been presented earlier. A working memory task was presented in between these two phases, and acted as a distracter task. During the first repetition of this sequence, the working memory task was a low-load 1-back task, and during the second repetition, the working memory task was a high-load 2-back task. A number of standard neuropsychological and subjective rating scales were also administered. Test sessions lasted about an hour.

Results of Underlying Drug Comparisons

Overall, LEV produced fewer untoward neuropsychological and neurophysiological effects than did CBZ in monotherapy at the dosages and timeframes employed in this study. Across all the standard neuropsychological tests, subjective rating scales, and cognitive neurophysiological tasks administered, significant differences were present for 42% (23 of 55) of the variables, all in favor of LEV; none favored CBZ. Compared to the non-drug average, CBZ was worse for 65% (36 of 55) and LEV was worse for 12% (4 of 33). Differential effects were seen for attention/vigilance, memory, language, psychomotor speed, graphomotor coding, reading/naming speed, subjective perceptions, and EEG neurophysiological measures. CBZ was associated with an increase in low frequency (<10 Hz) EEG power and changes in brain evoked potential (EP) measures. Linear discriminant analysis yielded highly accurate detection of treatment with CBZ relative to either LEV or non-drug conditions; detection was most accurate using the EEG together with an indirect neurophysiological measure.

Methodology for Predictive Analysis

Of the many standard neuropsychological tests that were significantly affected by CBZ, the Symbol Digits Modality Test (SDMT) had the largest effect (along with the Stroop) with an average decline of 6.7% (+/−3.21%) across the 28 subjects (p<0.0003), and was chosen as the primary dependent variable whose outcome was to be predicted from the non-drug baseline data.

The SDMT is a standard neuropsychological test of “executive function” and includes complex scanning, visual tracking, and agility components. These non-drug baseline values of task performance, EEG, and evoked potential variables that were most sensitive to the effects of CBZ were used as candidate independent predictor variables. Stepwise multiple linear regression analysis was used to identify which of these candidate independent predictor variables at baseline best predicted the change in the SDMT after taking CBZ.

Results

Neither non-drug baseline values of the SDMT, nor an IQ surrogate (Peabody Picture Vocabulary Test), nor a number of other standard neuropsychological test scores predicted the change in the SDMT after taking CBZ.

However, the analysis revealed that: a) the baseline values of an EEG variable, average power in the 2-10 Hz band measured at midline pariteo-occipital electrode POz during a staring-at-a-dot task, and b) a performance variable, reaction time in the syllables judgment task, significantly predicted (p<0.001) how much an individual's SDMT score would change after taking CBZ. Together these two variables accounted for 47% of the variance observed in SDMT score change.

Of the two variables, the EEG was the major contributor, by itself accounting for 30% of the variance (p<0.001). An average decline of 6.9%+/−2.21% in performance on the SDMT under CBZ treatment was predicted, as compared with the actual average decline of 6.7% (+/−3.21%). Six of the 28 subjects experienced a decline in SDMT score of at least 15% after taking CBZ. The predicted post-drug decline in the SDMT score was calculated for each of the 28 subjects using the regression equation based on the non-drug baseline values of reaction time and 2-10 Hz EEG power.

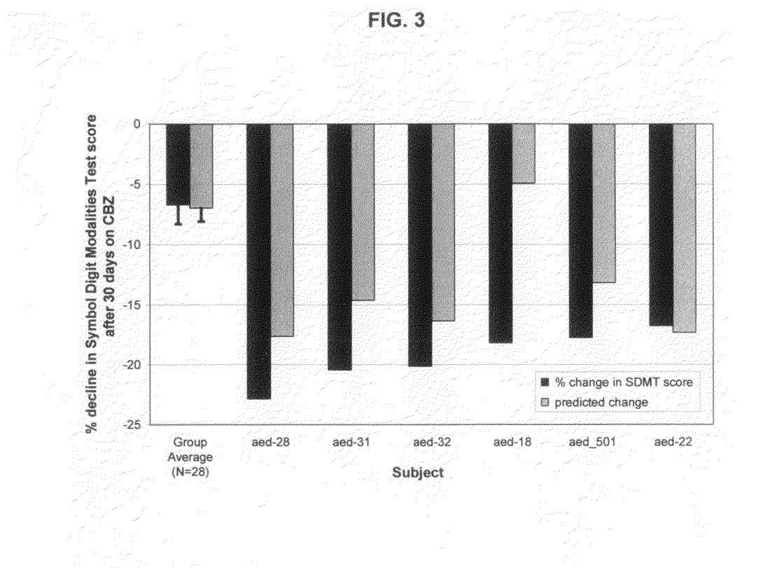

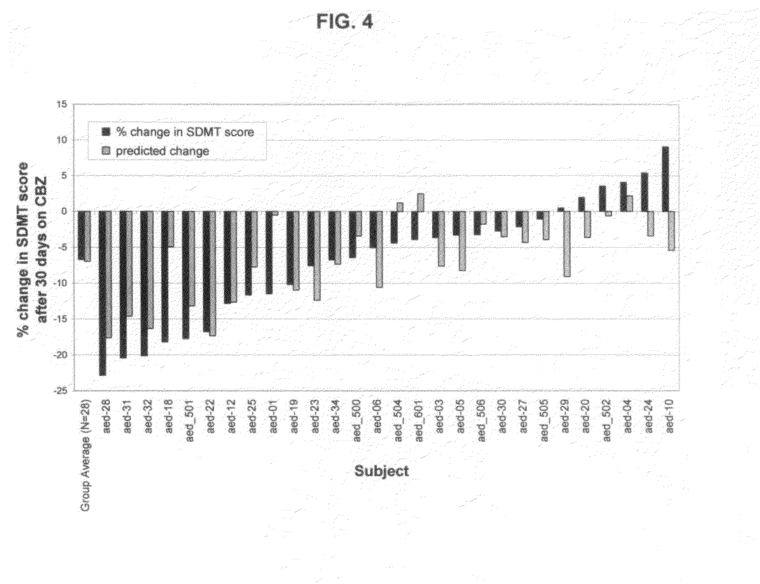

As is illustrated in FIG. 3, the regression equation predicted a decline in SDMT score of 13% or more for 5 out of the 6 subjects with the worst declines in SDMT scores. As is illustrated in FIG. 4, the regression equation significantly predicted the substantial range of individual differences in response to CBZ. Whereas the overall profile was negative, some subjects exhibited only mild neurocognitive side-effects, if any, while others became quite debilitated by CBZ.

When compared to the mean predicted SDMT change of 7% for all 28 subjects, these results showed that, based on measurements taken during a baseline state during which no drugs are administered, this method predicted in most cases when an individual is likely to suffer particularly negative cognitive side-effects if prescribed CBZ.

The two independent variables whose baseline values were found to predict change in SDMT score after taking CBZ, and similarly for related drugs, were EEG power from 2-10 Hz during an easy staring-at-a-dot task and reaction time in a syllable judgment task. Together these two variables accounted for 47% of the variance observed in SDMT score change. Of the two variables, the EEG was the major contributor, by itself accounting for 30% of the variance (p<0.001). With regard to the task performance variable, subjects who had longer reaction times on the syllable judgment task during their non-drug baseline test tended to have larger decreases in SDMT score after taking CBZ. It is unlikely that there is anything specific about the syllable judgment task that makes it particularly sensitive for such a prediction. Indeed, reaction times on other tasks employed in the study showed similar patterns, including working memory tasks.

This suggests that subjects who had more difficulty with the tasks under non-drug conditions may have relatively little excess “cerebral capacity” to absorb the effects of a cognitive stressor such as CBZ. However, standard neuropsychological test scores, including an IQ surrogate, did not significantly predict the post-drug decline in cognitive function.

With regard to the EEG variable, subjects who exhibited more 2-10 Hz EEG power tended to show a large decline in SDMT score after taking CBZ. It is unlikely that this effect is specific to the stare-at-a-dot task or to this precise EEG power band. The finding of greater broad-band EEG power (extending from 2 to well beyond 10 Hz into the beta band) during a non-drug baseline being associated with larger declines on SDMT scores was observed in other tasks as well.

Another analysis was how a subject responded to CBZ as correlated to a single baseline EEG variable, peak alpha frequency. Peak alpha frequency characterizes the dominant resonant frequency of large neuronal populations in the cerebral cortex and tends to be stable within a half Hertz in the same individual tested under the same conditions. It is sensitive to many factors that alter such resonance including mental tasks, alertness, drugs and illness, and is thus a sensitive nonspecific marker that a treatment or condition has altered central nervous system activity. Interestingly, this variable at baseline predicted the post-drug change in subjects' self-rated tiredness according to the SEALS battery, a widely used subjective symptom rating scale. The baseline EEG peak alpha frequency measure accounted for 18% of the variance in the change in the self-rated tiredness (p<0.03). This feeling of tiredness is not simply sleepiness, as this rating was neither correlated with ratings on the Karolinska sleepiness scale or neurophysiological measures of alertness. Rather, this report of “tiredness” may reflect a state of mental clouding and low volition Again, it is unlikely that peak alpha frequency is the only characteristic of the baseline EEG that is related to post-treatment subjective effects of the drug. Other EEG and task performance baseline predictors could include any of such measures sensitive to an individual's level of cognitive ability as described in Gevins and Smith, 2000.

The following sets forth variables that achieved the greatest prediction of drug response prior to drug administration for CBZ and related drugs:

1A. The baseline values of an EEG variable, average power in the 2-10 Hz band measured at midline pariteo-occipital electrode POz during an easy staring-at-a-dot task, and

1B. Reaction time in the syllables judgment task.

Table 3 sets forth results from this and other Examples herein; Table 3 uses the alphanumeric numbering used for results in this Example. Table 3 is grouped by functional category and sets forth findings at various levels of generality; these levels are each an embodiment of the invention. This Table exemplifies and does not limit the invention. Other embodiments, e.g., at other levels of generality or combining elements at various levels of generality will be apparent to those of skill in the art.

Discussion

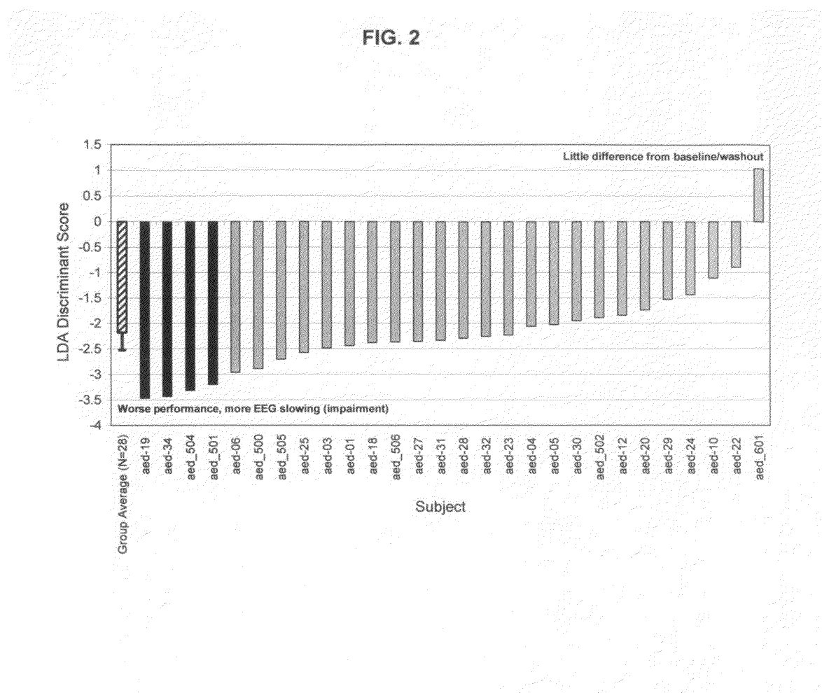

Prior to the present invention, there has been an unmet need of being able to predict whether a particular patient is likely to have adverse side effects before actually taking a particular drug. The results of this study indicated that there are substantial individual cognitive and neurophysiological differences in response to ingesting CBZ. Whereas the overall neurocognitive profile was negative, some subjects exhibited only mild neurocognitive side-effects, if any, while others became quite debilitated by CBZ (FIG. 2). Overall, it was possible to detect that an individual's neurocognitive function was affected by ingesting CBZ with 100% sensitivity and 100% specificity.

Accordingly, it was possible to predict who would have the most negative effects of CBZ based on measurements taken at non-drug baseline sessions. Specific latent characteristics were found to be predictive of a large negative reaction to CBZ. The, early identification of which patients should not be prescribed the drug is now possible.

In summary, this analysis showed that, in the case of a widely prescribed antiepileptic drug, it was possible to predict severe cognitive side-effects in an individual before they have taken the drug.

TABLE 3
Categorization of Parameters Used in Examples

| Measurement | | | | Example | |
|---|---|---|---|---|---|
| Category | Broader Subcategory | Parameter from Examples | Specific Parameter from Examples | Element | Example |
| EEG-Continuous | Banded spectral EEG | 2-10 Hz EEG power | Power in the 2-10 Hz band at | 1A | 1 |
| Activity | parameters | | midline pariteo-occipital | | |
| | | | electrode POz during an easy | | |
| | | | staring-at-a-dot task | | |
| EEG-Continuous | Banded spectral EEG | 2-20 Hz EEG power | Left-frontal 2-20 Hz EEG power | 3A | 3 |
| Activity | parameters | | in all tasks (relative | | |
| | | | weight 81), | | |
| EEG-Continuous | Banded spectral EEG | 2-10 Hz EEG power | Occipito-parietal 2-10 Hz EEG | 4A | 4 |
| Activity | parameters | | power in all tasks (relative | | |
| | | | weight .6) | | |
| EEG-Continuous | Banded spectral EEG | 6-20 Hz EEG power | Frontal and parietal relative | 6D | 6 |
| Activity | parameters | | 6-20 Hz EEG power | | |
| EEG-Continuous | Peak frequency in an | Alpha band peak frequency | Alpha band peak frequency at the | 2D | 2 |
| Activity | EEG band (e.g., | | right parietal electrode | | |
| | delta, theta, alpha, | | P4 (weight 0.59). | | |
| | beta, gamma) | | | | |
| EEG-Continuous | Peak frequency in an | Alpha band peak frequency | Peak alpha frequency in all | 4B | 4 |
| Activity | EEG band (e.g., | | tasks (relative weight .3) | | |
| | delta, theta, alpha, | | | | |
| | beta, gamma) | | | | |
| Task | Reaction time | Reaction time during an attention | Reaction time in syllables | 1B | 1 |
| Performance | | demanding task | judgment task | | |
| Task | Reaction time | Mean reaction time-during an | Mean working memory task | 2A | 2 |
| Performance | | attention demanding task | reaction time (weight-0.26) | | |
| Task | Reaction time | Reaction time during an attention | Reaction time during a syllable | 5B | 5 |
| Performance | | demanding task | counting task (relative weight | | |
| | | | 60) | | |
| Task | Accuracy of attention | Performance accuracy in a | Performance accuracy in the 2- | 3C | 3 |
| Performance | demanding task | working memory task | back working memory task | | |
| | response | | (relative weight 34). | | |
| Task | Accuracy of attention | Performance accuracy in an | Episodic and working memory task | 6A | 6 |
| Performance | demanding task | episodic memory task | performance accuracy and | | |
| | response | | reaction time | | |
| EEG-Evoked | Cognitively-modulated | Evoked potential slow wave | Evoked potential slow wave | 2C | 2 |
| Potential | evoked potential | amplitude, size or shape or timing | amplitude (400 to 600 ms) at the | | |
| | | | right parietal electrode P4 | | |
| | | | (weight 0.56); | | |
| EEG-Evoked | Cognitively-modulated | P300 evoked potential amplitude, | Parieto-occipital P300 evoked | 3B | 3 |
| Potential | evoked potential | size, shape or timing | potential amplitude during a | | |
| | | | 1-back working memory task | | |
| | | | (relative weight 45) | | |
| EEG-Evoked | Cognitively-modulated | P200 evoked potential amplitude, | P200 evoked potential amplitude | 5A | 5 |
| Potential | evoked potential | size, shape or timing | during an episodic memory task, | | |
| | | | (relative weight 50), | | |
| EEG-Evoked | Cognitively-modulated | CNV evoked potential amplitude, | Frontal and parietal CNV and | 6B | 6 |
| Potential | evoked potential | size or shape or timing | late positive slow wave evoked | | |
| | | | potential amplitude | | |
| EEG-Evoked | Cognitively-modulated | Evoked potential slow wave | Evoked potential slow wave | 2B | 2 |
| Potential* | evoked potential | amplitude, size or shape or timing | amplitude (300 to 800 ms) at | | |
| | | | the right frontal electrode F4 | | |
| | | | (weight 0.65); | | |
| Genetic | Genetic marker | Protein genetic marker | ApoE4 genetic marker | 6C | 6 |
| Information | | | | | |
| Self Report | Subject's self- | Self-rated fatigue rating on a | Fatigue rating on the POMS scale | 4C | 4 |
| | assessment of their | structured scale | (relative weight .3) | | |
| | condition (e.g., | | | | |
| | affective, cognitive | | | | |
| | and alertness state) | | | | |
| Self Report | Subject's self- | Self-rated cognitive rating on a | Cognition rating on the SEALS | 5C | 5 |
| | assessment of their | structured scale | scale (relative weight 71). | | |
| | condition (e.g., | | | | |
| | affective, cognitive | | | | |
| | and alertness state) | | | | |

*Including cognitive and sensory, evoked potentials in auditory, visual & somatosensory modalities, and response-related evoked potentials

### Example 2

### Prediction of Effective Drug Dose

The following experiment determined the optimal dose of the commonly prescribed psychostimulant drug methylphenidate for treating Attention Deficit Hyperactivity Disorder (ADHD) from test doses. The diagnosis of ADHD is defined in the DSM IV-TR (Diagnostic and Statistical Manual of Mental Disorders). The ADHD diagnosis identifies characteristics such as hyperactivity, forgetfulness, mood swings, poor impulse control, and distractibility, as symptoms of an unspecified underlying neurological pathology.

This study assessed the sensitivity of data obtained in accordance with the present invention in evaluating varying doses of methylphenidate (MPH, Ritalin™) in treating pediatric ADHD. The analysis described herein aimed at determining whether the SAM Exam could match the optimal dose of methylphenidate that was independently selected by a pediatric psychiatrist specialist who prescribed the drug in accordance with current methods in the art.

Subjects

Fourteen patients diagnosed with ADHD and clinically classified by a physician specialist in ADHD as MPH-responders (patients who would benefit from taking MPH) completed the protocol (11 males, 3 females; age range 8-18, mean 11.3, standard deviation 3.1). All patients weighed more than 25 kg.

Protocol

Patients received one week each of placebo (0 mg), 5 mg, 10 mg, 15 mg, and 20 mg daily doses of MPH for a total of five weeks, according to a fully counterbalanced, placebo-controlled, double-blind design. In this example EEG data was obtained concurrent with selected attention demanding tasks. This data was obtained in accordance with procedures set forth in U.S. Pat. Nos. 6,947,790, 6,434,419 and 5,295,491. Accordingly, an EEG computer-based examination was obtained simultaneously with the subject taking a test battery of attention demanding tasks, for example a set of psychometric tasks. Such data was obtained once a week, 1-3 hours post-ingestion of the prescribed dose for that week. Examinations were administered approximately the same time of day and on the same day of the week during the dose titration period. The protocol used in this study included a low-load simple reaction time task (SRT) task and a higher-load 1-back working memory (WM) task in sessions lasting about half an hour.

Treatment effects were assessed at the end of each titration week with rating scales, including Strengths and Weaknesses of ADHD-symptoms and Normal-behaviors (SWAN) and side effect ratings from schoolteachers and parents. These scales were then assessed by a clinician, in accordance with current methods in the medical art, to determine the dose for each patient at the end of the 5-week dose titration period.

Data in Accordance with the Invention Compared to Prior Analysis

Prior multivariate analyses of the clinical and parent/teacher rating scales, using a repeated measures ANOVA with dose (5 levels) as a within-subject factor, did not reveal any systematic dose-response relationship. The data obtained in accordance with the present invention readily detected the presence of MPH treatment (average of all doses) relative to placebo (p<0.001), but did not reveal any systematic dose-response relationship across patients.

Inspection of the EEG and performance data suggested that there were clear maxima at doses that varied across patients. This analysis was used to determine the minimum dose necessary for a child to reach a ceiling in improved psychometric task performance and EEG markers of improved attention; these findings were evaluated to see how well these dosages related to a pediatric psychiatrist's recommended dose.

Analysis focused on the 1-back working memory task whose performance and EEG signals were most affected by MPH A divergence-based multivariate feature selection and pattern classification algorithm (for convenience called the neuroworkload meter—Gevins and Smith, 2003; Smith, Gevins, et al, 2001) was employed to choose and weight an optimal subset of features to characterize the MPH response.

This algorithmic approach to optimal feature subset selection has been applied by the inventor to many other drugs and treatment conditions, including anti-epileptic drugs such as topiramate, carbamazepine, lamotrigine and levitiracetam, antihistamine drugs such as diphenhydramine, benzodiazepine class drugs such as alprazolam, analgesic drugs of the canabanoid class, other psychostimulants such as caffeine, intoxicants such as alcohol and marijuana, mild cognitive impairment due to sleep loss and amnestic mild cognitive impairment characterizing incipient Alzheimer's disease.

The inventors have also used this method to predict accidents during simulated driving.

Results

A set of candidate 1-back WM performance and attention-sensitive EEG features most affected by varying MPH doses was selected based on the prior statistical analyses of the average of the MPH doses followed by visual inspection of the grand averaged EEG power spectra and evoked potential data. Feature subsets were limited to a maximum of four variables because of the small number of subjects in the experiment.

The following set of four task variables achieved the greatest separation of all the MPH doses, and related drugs, from placebo:

2A. Mean working memory task reaction time (weight −0.26);

2B. Evoked potential slow wave amplitude (300 to 800 ms) at the right frontal electrode F4 (weight 0.65);

2C. Evoked potential slow wave amplitude (400 to 600 ms) at the right parietal electrode P4 (weight 0.56);

2D. EEG alpha band peak frequency at the right parietal electrode P4 (weight 0.59).

Table 3 sets forth results from this and other Examples herein; Table 3 uses the alphanumeric numbering used for results in this Example. Table 3 is grouped by functional category and sets forth findings at various levels of generality; these levels are each an embodiment of the invention. This Table exemplifies and does not limit the invention. Other embodiments, e.g., at other levels of generality or combining elements at various levels of generality will be apparent to those of skill in the art.

Decreased reaction time and increased EP slow wave amplitudes are all markers of improved attention A change in alpha peak frequency is an indicator that a drug is affecting the central nervous system. Of the four variables, the two evoked potential slow wave amplitude measures had the greatest weighting in the equation.

However, as appreciated by those skilled in the art, these variables and their exact or relative weightings are not unique representations of the neurophysiologic attentional processes or their behavioral manifestations that are affected by MPH. Other variables, with other relative and absolute weightings, that characterize performance of attention demanding tasks and the neural regulation of such performance in frontal, parietal and other brain regions can also be extracted using the same methodology on different sets of data. Examples of such sets of variables are described in the inventor's prior patents and scientific publications referred to herein. While they are not unique, as noted, the choice, combination and weighting of these four variables do not merely reflect the variance in the particular MPH data that were analyzed. This set of variables also characterizes other psychostimulant drugs and other classes of drugs that affect attention and alertness including both alerting and sedating drugs. This is so because drugs that affect attention and alertness impact the accuracy and speed of performance of attention demanding tasks and such attention-demanding performance is mediated by neuronal processes in frontal and parietal cerebral cortex that are indexed by evoked potential slow wave measures and the peak frequency of the alpha rhythm.

An equation to distinguish the 15 mg dose from placebo was computed by a divergence-based multivariate feature selection and pattern classification algorithm (for convenience called the neuroworkload meter—Gevins and Smith, 2003; Smith, Gevins, et al, 2001) using these four variables.

The sensitivity and specificity of this equation were evaluated for the various doses and were highly significant in all cases. For instance, sensitivity was 86% and specificity was 93% in distinguishing 20 mg from placebo (p<0.0001), effect size 2.12 (Cohen's d), area under the ROC curve 0.923 (p<0.0001).

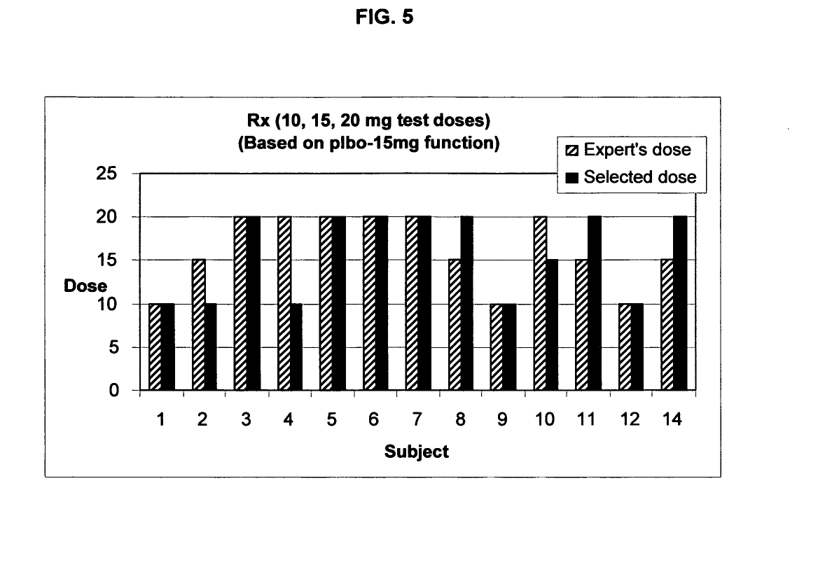

For each subject, the highest value output by this equation for doses of 10, 15 and 20 mg was taken to be the recommended dose to be compared with the dose chosen by the pediatric specialist. (A 5 mg dose was not considered because the specialist only chose that dose for a single atypical patient out of the 14 patients; that patient was not included in the analysis). For 12 out of 13 patients the dose determined in accordance with the present invention (“selected dose”) agreed with the pediatric physician's dose within 5 mg (p<0.01, binomial test) (FIG. 5). These results are from one embodiment of a clinical test where three test doses and three assessments were administered.

In an alternative embodiment, determination of the best dose for a patient is based on the response to a single test dose. For instance we used a test dose of 15 mg in a linear regression to predict the patient's response to 10 or 20 mg. For MPH this was less preferred since the response to MPH is non-linear and the function appears to differ amongst patients. Since each patient responds somewhat differently to varying doses of MPH, it is quite difficult to determine in which direction the dose should be adjusted based on test data in accordance with the invention corresponding to a single test dose. However, in the context of MPH, use of two test doses to predict the physician's dose is more preferred, and is clinically practical with two visits; this is a marked improvement over the current art where the clinician must often test an entire range of doses.

Determination of the best dose for a patient based on the response to a single test dose is a preferred embodiment of the invention for those drugs that have a linear response to dose, and limited patient to patient variability. Examples of drug classes and drugs with linear response characteristics and limited variability across patients include diazepines, benzodiazepines, alprazolam, diazepam, clonazepam, barbiturates, phenobarbital, pentobarbital and many other sedating drugs.

Table 2 shows data from use of the invention to predict the pediatric specialist's dose with various combinations of two doses. The prediction was simply made by considering the dose with the maximum response and the slope between the responses to the two doses. Of the combinations assessed, test doses of 10 and 20 mg were the best in that the specialist's dose was matched for 7 patients, and the direction of increasing or decreasing dose from the test doses was correctly indicated in 4 of the other 6 patients. Using this two-dose test approach, the data in accordance with the invention matched the physician specialist's dose within 5 mg for 11 of 13 patients (p<0.05, binomial test). The dose with the highest score from the above analysis was greater than the score on placebo with an effect size of 3.43 (Cohen's d).

Discussion

The results of this analysis indicate that a good therapeutic dose of a drug such as methylphenidate to treat ADHD can be determined from as few as two test doses.

TABLE 2

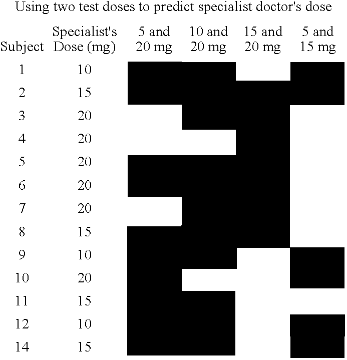

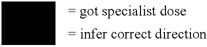

### Example 3

### Prediction of Drug Response Prior to Administering a Drug

This example sets forth predictions from data prior to drug administration. Accordingly, from a non-drug baseline, the health care provider determines whether a subject will have a positive or an adverse neurocognitive response to the common anti-epileptic drug topiramate.

In Smith, Gevins, Meador, et al., 2006, the cognitive neurophysiological effects of topiramate were examined in a double-blind, randomized, crossover design. Principally, topiramate adversely affected working memory task performance and increased 2-6 Hz EEG power. In the present analysis, we predict subjects' neurocognitive response to topiramate from her or his non-drug baseline data. To this end, we first computed how much each subject's neurocognitive function was affected by taking topiramate, grouping them as “bad responders” and “OK responders.” Accordingly, an array of direct and indirect brain function measures that differed between “bad responders” and “OK responders” was compiled from the non-drug baseline data.

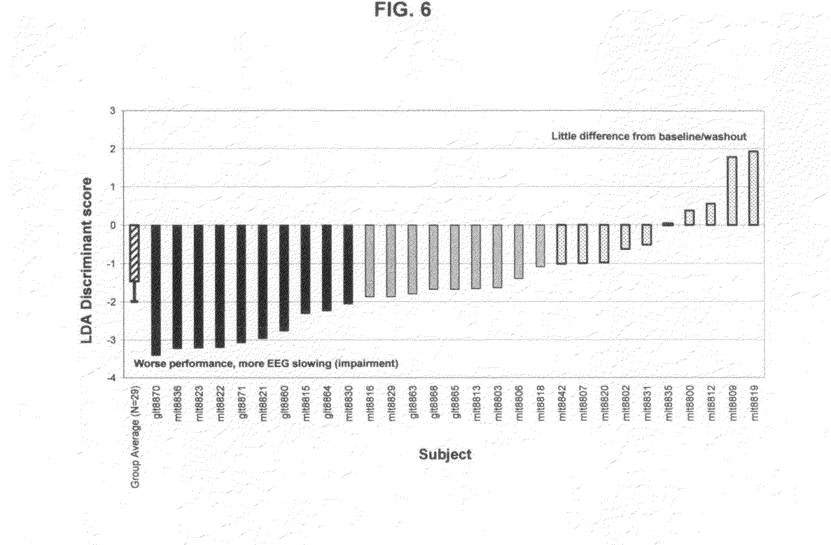

First, a set of working memory task performance and EEG variables found to differ between post-topiramate and pre-topiramate baseline in the above-referenced study was entered into a stepwise linear discriminant analysis (LDA) in order to generate a score for each of the 29 subjects, quantifying his or her neurocognitive response to the drug (FIG. 6). From this data we then formed two groups of 10 subjects, called “bad responders” and “OK responders,” with the lowest and highest LDA scores, respectively.

Non-drug baseline EEG and evoked potential data from the two groups were analyzed to identify which variables differed most between “bad responders” and “OK responders.” These variables, plus task performance variables from the above-referenced study, were considered to be candidate predictor variables in an analysis to predict each subject's neurocognitive response to topiramate from the subject's non-drug baseline data.

A divergence-based multivariate feature selection and pattern classification algorithm (for convenience called the neuroworkload meter—Gevins and Smith, 2003; Smith, Gevins, et al, 2001) was then applied to choose and weight an optimal subset of three candidate predictor variables at non-drug baseline to distinguish between “bad responders” and “OK responders.” The three final predictor variables were left-frontal 2-20 Hz EEG power in all tasks (relative weight 81), parieto-occipital P300 evoked potential amplitude during a 1-back working memory task (relative weight 45), and performance accuracy in the 2-back working memory task (relative weight 34). The “bad responders” were distinguished from the “OK responders” with a sensitivity of 100% and a specificity of 80%.

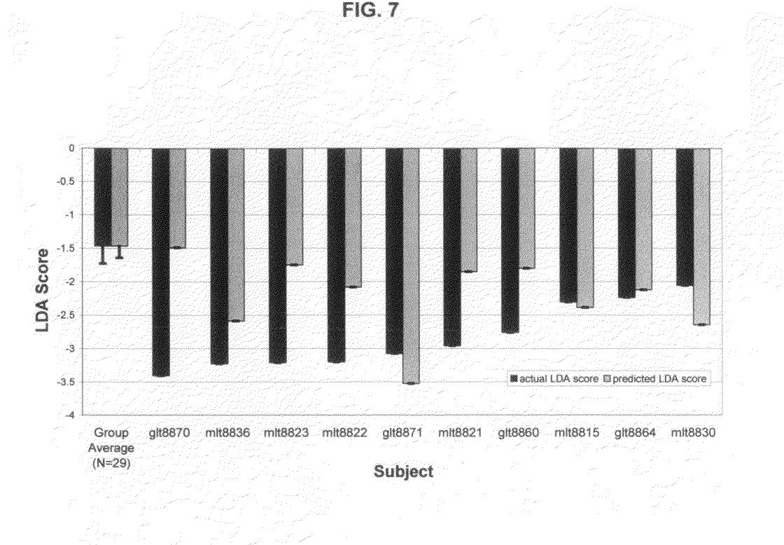

Finally, the three final weighted predictor variables were entered into a stepwise linear regression to predict each subject's LDA neurocognitive drug response score. The results were highly significant (p<0.003). The about equally weighted non-drug baseline 2-20 Hz EEG power and the P300 amplitude evoked potential variables collectively accounted for 44% of the variance in the post-drug LDA score. These results showed that such non-drug baseline brain function measures can be used to predict the magnitude of a subject's response to topiramate (FIG. 7), and related drugs.

In non-drug baseline conditions, “bad responders” had lower 2-20 Hz EEG power, higher P300 amplitude, and higher working memory task performance accuracy. In the inventor's U.S. Pat. No. 6,434,419 and in Gevins and Smith, 2000, it was documented that subjects with higher IQ scores had a similar constellation of findings. The current results thus suggest that subjects with greater neurocognitive ability were most debilitated by topiramate.

This is quite in contrast with the direction of similar brain function variables that predicted neurocognitive response to carbamazepine (Example 1). For carbamazepine, the results suggested that subjects with lower neurocognitive capacity would be most adversely affected. The mechanism of action of topiramate is quite different from that of carbamazepine, suggesting that differing non-drug baseline patterns of direct and indirect brain function measures are predictive of responses to different types of anti-epileptic drugs. With regard to the particular brain function variables found to be predictive of topiramate's neurocognitive effect, comments similar to those made in Example 1 apply, i.e. the variables found here would apply to other drugs or treatments that affect an individual's cognitive ability, and other combinations of brain function variables that characterize cognitive ability would be affected by topiramate.

Thus, taken from a pre-drug baseline, the following set of variables achieved the greatest predictive value for topirimate and related drug outcome:

3A. Left-frontal 2-20 Hz EEG power in all tasks (relative weight 81),

3B. Parieto-occipital P300 evoked potential amplitude during a 1-back working memory task (relative weight 45)

3C. Performance accuracy in the 2-back working memory task (relative weight 34).

Table 3 sets forth results from this and other Examples herein; Table 3 uses the alphanumeric numbering used for results in this Example. Table 3 is grouped by functional category and sets forth findings at various levels of generality; these levels are each an embodiment of the invention. This Table exemplifies and does not limit the invention. Other embodiments, e.g., at other levels of generality or combining elements at various levels of generality will be apparent to those of skill in the art.

### Example 4

### Direct Brain Function Measures and Indirect Brain Function Measures (Such as Patient Reporting) to Accurately Determine Drug Response

This example sets forth the measurement of response after drug administration using a combination of direct and indirect brain function measures; in this embodiment, EEG direct measures and subject's subjective reports were the indirect measures. Accordingly, the health care provider determines whether a subject has had a positive or an adverse neurocognitive response to the common anti-epileptic drug carbamazepine.

Using the data and methods described in Example 1, we determined how a combination of post-drug EEG and the subject's subjective scale measures quantifies the response to carbamazepine. Accordingly, a stepwise linear discriminant analysis (LDA) was used to generate a neurocognitive drug response score for each subject, reflecting the magnitude of neurocognitive response to the drug as compared with the non-drug baseline. The sensitivity and specificity in detecting the effect of the drug were both 100%.

Three variables were used in the LDA, of which two were EEG and one was a subjective scale measure. The EEG variables were occipito-parietal 2-10 Hz EEG power in all tasks (relative weight 0.6) which increased consequent to CBZ, peak alpha frequency in all tasks (relative weight 0.3) which decreased consequent to CBZ, and self-reported fatigue rating on the Profile of Mood Scale (POMS, Jacobson et al., 1978) (relative weight 0.3) which increased consequent to CBZ.

The following set of variables quantified the response to carbamazepine and related drugs:

4A. Occipito-parietal 2-10 Hz EEG power in all tasks (relative weight 0.6),

4B. Peak alpha frequency in all tasks (relative weight 0.3)

4C. Self-reported fatigue rating on the Profile of Mood Scale (POMS, Jacobson et al., 1978) (relative weight 0.3).

Table 3 sets forth results from this and other Examples herein; Table 3 uses the alphanumeric numbering used for results in this Example. Table 3 is grouped by functional category and sets forth findings at various levels of generality; these levels are each an embodiment of the invention. This Table exemplifies and does not limit the invention. Other embodiments, e.g., at other levels of generality or combining elements at various levels of generality will be apparent to those of skill in the art.

### Example 5

### Prediction of Drug Response Prior to Administering a Drug from Direct Brain Function Measures and Indirect Brain Function Measures (Such as Task Performance and Patient Reporting)

This example sets forth drug response determinations from data prior to administering the drug. A combination of direct and indirect brain function measures were used to make the determinations, namely EEG direct measures and task performance and subject's subjective report as the indirect measures. Accordingly, from a non-drug baseline, the health care provider determines whether a subject will have a positive or an adverse neurocognitive response to the common anti-epileptic drug carbamazepine.

Using the data and methods described in Example 1, we determined how a combination of non-drug baseline EEG, task performance and the subject's subjective scale measures predict the Symbol Digit Modalities Test (SDMT) cognitive drug response outcome measure.

Accordingly, we first formed two groups of 10 subjects, called “bad responders” and “OK responders,” with the highest and lowest declines in SDMT scores after taking carbamazepine (respectively the 10 leftmost and 10 rightmost subjects in FIG. 4). In order to predict each subject's neurocognitive response to carbamazepine from the subject's non-drug baseline data, non-drug baseline EEG, evoked potential, task performance and subjective scale data from the two groups were iteratively analyzed to identify candidate predictor variables which differed most between the “bad responders” and the “OK responders.” A divergence-based multivariate feature selection and pattern classification algorithm (for convenience called the neuroworkload meter—Gevins and Smith, 2003; Smith, Gevins, et al, 2001) was then applied to choose and weight an optimal subset of three candidate predictor variables at non-drug baseline to distinguish between “bad responders” and “OK responders.”

The three final predictor variables were P200 evoked potential amplitude during an episodic memory task, (relative weight 50), reaction time during a syllable counting task (relative weight 60), and self-rated cognition on the Side Effects and Life Satisfaction scale (SEALS, Gillham et al., 1996) (relative weight 71). The “bad responders” were distinguished from the “OK responders” with a sensitivity of 60% and a specificity of 100%. Subjects with smaller P200 amplitudes, longer reaction times and higher SEALS scores at baseline had the worst effects. As in Example 1, all three variables are consistent with relatively lower cognitive ability. A smaller P200 during the episodic memory task is associated with reduced attention; longer reaction times imply poorer performance; and subjects with high SEALS-cognition scores believe they are not thinking clearly.

These findings show that combining direct EEG evoked potential and indirect self-report and task performance brain function measures serves to predict the neurocognitive effects of drugs.

The following set of variables serves to determine whether a subject will have a positive or an adverse neurocognitive response to carbamazepine and related drugs:

5A. P200 evoked potential amplitude during an episodic memory task (relative weight 50),

5B. Reaction time during a syllable counting task (relative weight 60), and

5C. Self-rated cognition on the SEALS scale (relative weight 71).

Table 3 sets forth results from this and other Examples herein; Table 3 uses the alphanumeric numbering used for results in this Example. Table 3 is grouped by functional category and sets forth findings at various levels of generality; these levels are each an embodiment of the invention. This Table exemplifies and does not limit the invention. Other embodiments, e.g., at other levels of generality or combining elements at various levels of generality will be apparent to those of skill in the art.

### Example 6

### Use of Genetic Marker Information in Combination with Task Performance and EEG Direct Brain Function Measures to Detect Mild Cognitive Impairment (MCI)

This analysis was performed to determine how combining genetic marker indirect brain function measures with task performance indirect brain function measures and EEG direct brain function measures improves the ability to detect amnestic mild cognitive impairment (MCI), the precursor to Alzheimer's Disease in elderly subjects.

A divergence-based multivariate feature selection and pattern classification algorithm (for convenience called the neuroworkload meter—Gevins and Smith, 2003; Smith, Gevins, et al, 2001) was used, as described above in Examples 2 and 3. Accordingly, a combination of direct (frontal and parietal CNV and late positive slow wave evoked potential, and relative 6-20 Hz EEG power) and indirect (episodic memory and working memory task performance accuracy and reaction time) brain function measures was capable of detecting probable mild cognitive impairment with good accuracy in a sample of 64 elderly adults. Detection accuracy was lower either using only the indirect attention demanding task performance measures, or only the direct EEG and evoked potential measures, or only the indirect generic marker information described below. The novel combination of direct and indirect brain function measures are synergistically predictive.

In addition, it is noted that individuals with the ApoE genetic marker were 2.77 times more likely to progress to Alzheimer's Disease (AD) than individuals without the marker, and are more likely to progress to MCI before progressing to AD. Therefore, detection of probable MCI is underestimated when such genetic information is not taken into account. In order to ascertain the amount by which sensitivity would increase when taking into account genetic information, a test in which EEG was recorded during cognitive testing was administered to a group of 26 older adults with the ApoE4 genetic marker. An analysis using memory task performance and evoked potential EEG measures identified 5 out of the 26 subjects (19%) as having probable amnestic MCI. Taking the genetic information into account by considering the increased risk of AD in subjects with the ApoE genetic marker resulted in identifying 8 out of 26 subjects (31%) as having probable amnestic MCI. Based on this assessment, genetic marker information along with other indirect and direct measures of brain function can be used synergistically in detecting amnestic mild cognitive impairment.

Accordingly, the following set of variables achieved the greatest ability to detect probable amnestic mild cognitive impairment (MCI), the precursor to Alzheimer's Disease:

6A. Episodic and working memory task performance accuracy and reaction time,

6B. Frontal and parietal CNV and late positive slow wave evoked potential amplitude,

6C. ApoE genetic marker,

6D. Frontal and parietal relative 6-20 Hz EEG power.

Table 3 sets forth results from this and other Examples herein; Table 3 uses the alphanumeric numbering used for results in this Example. Table 3 is grouped by functional category and sets forth findings at various levels of generality; these levels are each an embodiment of the invention. This Table exemplifies and does not limit the invention. Other embodiments, e.g., at other levels of generality or combining elements at various levels of generality will be apparent to those of skill in the art.

### CITATIONS

Meador, K. J., Gevins, A., Loring, D. W., McEvoy, L. K., Ray, P. G., Smith, M. E., Motamedi, G. K., Evans, B. M. and Baum, C. (2007) Neuropsychological and Neurophysiological Effects of Carbamazepine and Levetiracetam. Neurology, 69:2076-2084.
McEvoy, L. K., Smith, M. E., Fordyce, M., Gevins, A. (2006) Characterizing impaired functional alertness from diphenhydramine in the elderly with performance and neurophysiologic measures. Sleep. 29, 959-966.
Smith, M. E., Gevins, A., McEvoy, L. K., Meador, K. J., Ray, P. G., & Gilliam, F. (2006) Distinct cognitive neurophysiologic profiles for lamotrigine and topiramate. Epilepsia, 47 (4), 1-9.
Ilan, A. B., Gevins, A., Coleman, M., ElSohly, M. A., & de Wit, a (2005). Neurophysiological and subjective profile of marijuana with varying concentrations of cannabinoids. Behavioural Pharmacology, 16, 487-96.
Gevins, A & Smith, M. E (2003). Neurophysiological measures of cognitive workload during human-computer interaction Theoretical Issues in Ergonomic Science, 4, 113-131.
Ilan A B, Smith M E, Gevins A (2004) Effects of marijuana on neurophysiological signals of working and episodic memory. Psychopharmacolog (Berl). 176, 214-222.
Smith, M. E., McEvoy, L. K., & Gevins, A (2002). The impact of moderate sleep loss on neurophysiologic signals during working memory task performance. Sleep, 25, 784-794.
Chung, S. S., McEvoy, L. K., Smith, M. E., Gevins, A., Meador, K. & Laxer, K. D. (2002). Task related EEG & ERP changes without performance impairment following a single dose of phenytoin. Clinical Neurophysiology, 113, 806-814.
Gevins, A., Smith, M. E., & McEvoy, L. K (2002). Tracking the cognitive pharmacodynamics of psychoactive substances with combinations of behavioral and neurophysiological measures. Neuropsychopharmacology, 26, 27-39.
Smith, M. E., Gevins, A., Brown, H., Karnik, A., & Du, R (2001). Monitoring task load with multivariate EEG measures during complex forms of human computer interaction Human Factors, 43, 366-380.
Ilan, A. B., & Gevins, A (2001). Prolonged neurophysiological effects of cumulative wine drinking. Alcohol, 25, 137-152.
McEvoy, L. K., Pellouchoud, E., Smith, M. E., & Gevins, A (2001). Neurophysiological signals of working memory in normal aging Cognitive Brain Research, 11, 363-376.
Gevins, A., & Smith, M. E (2000). Neurophysiological measures of working memory and individual differences in cognitive ability and cognitive style. Cerebral Cortex, 10, 829-839.
McEvoy, L. K., Smith, M. E., & Gevins, A. (2000). Test-retest reliability of task-related EEG. Clinical Neurophysiology, 1, 457-463.
Pellouchoud, E., Smith, M. E., McEvoy, L., & Gevins, A. (1999). Mental effort related EEG modulation during video game play: Comparison between juvenile epileptic and normal control subjects. Epilepsia, 40, Supple 4: 38-43.
Gevins, A., & Smith, M. E (1999). Detecting transient cognitive impairment with EEG pattern recognition Aviation, Space, and Environmental Medicine, 70, 1018-1024.
Smith, M. E., McEvoy, L., & Gevins, A (1999). Neurophysiological indices of strategy development and skill acquisition Cognitive Brain Research, 7, 389-404.
McEvoy, L Smith, M. E & Gevins, A (1998) Dynamic cortical networks of verbal and spatial working memory. Cerebral Cortex, 8, 563-574.
Gevins, A., Smith, M. E., Leong, H., McEvoy, L., Whitfield, S., Du, R., & Rush, G. (1998). Monitoring working memory load during computer-based tasks with EEG pattern recognition Human Factors, 40, 79-91.
Gevins, A., Smith, M. E., McEvoy, L., & Yu, D. (1997). High resolution EEG mapping of cortical activation related to working memory: Effects of task difficulty, type of processing, and practice. Cerebral Cortex, 7, 374-385.
Gillham R, Baker G, Thompson P, Birbeck K, McGuire A, Tomlinson L, Eckersley L, Silveira C, and Brown S (1996). Standardisation of Self-report Questionnaire for Use in Evaluating Cognitive, Affective and Behavioral Side-effects of Antiepileptic Drug Treatments. Epilepsy Research, 24, 47-55.

1. A method of predicting response to a drug prior to drug administration comprising:
obtaining a direct brain function measurement;
obtaining an indirect brain function measurement; and,
assessing the direct brain function measurement and the indirect brain function measurement collectively to obtain a drug response prediction,
whereby the predictive value of the collective assessment is greater than a predictive value obtained from the separate predictive values for the direct and indirect measurements.

2. The method according to claim 1 wherein the direct brain function measurement and the indirect brain function measurement are selected from an example set forth in Table 3.

3. The method according to claim 1 wherein the direct brain function measurement and the indirect brain function measurement elicit data on the same physiological functions as set forth in an example represented in Table 3.

4. The method according to claim 3 wherein the physiological functions are selected from the group consisting of: attention regulation, memory, alertness regulation, regulation of other neurocognitive functions, regulation of sensory or motor functions, and regulation of mass neuronal synchronization.

5. A method of determining dosage of a drug with a linear dose-response curve, comprising steps of:
administering a drug;
obtaining a direct brain function measurement;
obtaining an indirect brain function measurement; and,
assessing the direct brain function measurement and the indirect brain function measurement collectively to obtain a dosage prediction
whereby the predictive value of the collective assessment is greater than a predictive value obtained from the separate predictive values for the direct and indirect measurements.

6. The method according to claim 5, wherein the direct brain function measurement and the indirect brain function measurement are selected from an example set forth in Table 3.

7. The method according to claim 5 wherein the direct brain function measurement and the indirect brain function measurement elicit data on the same physiological functions as set forth in an example represented in Table 3.

8. The method according to claim 7 wherein the physiological functions are selected from the group consisting of: attention regulation, memory, alertness regulation, regulation of other neurocognitive functions, regulation of sensory or motor functions, and regulation of mass neuronal synchronization.

9. A method of determining dosage of a drug with a nonlinear dose-response curve to achieve a specified response, comprising steps of:
administering a first test dose of the drug;
calculating a dose-response value for the test dose; subsequently,
administering a second test dose of the drug;
calculating a dose-response value for the second test dose;
calculating the slope between the responses to the two doses;
extrapolating from the slope to a reach a specified response level; and,
identifying the dose that corresponds to that response level.

10. The method according to claim 9 wherein the indirect measurement is selected from the group consisting of: information about brain structure, information from genetic measures, information from bodily fluids, information about a patient's behavior from task performance data, psychometric data, self-report data, third party assessment and clinical scales.

11. A method of detecting mild cognitive impairment (“MCI”), comprising steps of:
obtaining a direct brain function measurement;
obtaining an indirect brain function measurement;
assessing the direct brain function measurement and the indirect brain function measurement collectively to obtain a prediction of the subject's brain function
whereby the predictive value of the collective assessment is greater than a predictive value obtained from the separate predictive values for the direct and indirect measurements.

12. The method of claim 11, wherein the step of obtaining an indirect brain function measurement comprises obtaining patient information on genetic marker for Apolipoprotein E and the assessing step comprises assessment of Apolipoprotein E.

13. The method of claim 12, wherein the step of obtaining an indirect brain function measurement comprises obtaining patient information on genetic marker for Apolipoprotein E and at least one other indirect measurement, and the assessing step comprises assessment of Apolipoprotein E and at least one other indirect measurement.

14. The method according to claim 11, wherein the direct brain function measurement and the indirect brain function measurement are selected from an example set forth in Table 3.

15. The method according to claim 11 wherein the direct brain function measurement and the indirect brain function measurement elicit data on the same physiological functions as set forth in an example represented in Table 3.

16. The method according to claim 15 wherein the physiological functions are selected from the group consisting of: attention regulation, memory, alertness regulation, regulation of other neurocognitive functions, regulation of sensory or motor functions, and regulation of mass neuronal synchronization.
